(19) United States
(12) Patent Application Publication
(10) Pub. No.: US 2025/0205155 A1
(43) Pub. Date: Jun. 26, 2025

(54) FORMULATIONS COMPRISING MELPHALAN FLUFENAMIDE AND CYCLODEXTRIN

(71) Applicant: Oncopeptides Innovation AB, Stockholm (SE)

(72) Inventors: Fredrik LEHMANN, Stockholm (SE); Peter TEODOROVIC, Stockholm (SE); Kristin HAMMER, Stockholm (SE); Rune RINGOM, Uppsala (SE)

(73) Assignee: Oncopeptides Innovation AB, Stockholm (SE)

(21) Appl. No.: 18/849,977

(22) PCT Filed: Mar. 24, 2023
(86) PCT No.: PCT/EP2023/057729

§ 371 Date: Sep. 23, 2024

(30) Foreign Application Priority Data
Mar. 24, 2022 (GB) 2204171.9

(51) Int. Cl. A61K 9/08; A61K 9/19; A61K 31/198; A61K 45/06; A61K 47/10; A61K 47/26; A61K 47/40

(52) U.S. Cl. CPC A61K 9/08; A61K 9/19; A61K 31/198; A61K 45/06; A61K 47/10; A61K 47/26; A61K 47/40

(57) ABSTRACT

The invention provides a pharmaceutical preparation comprising melphalan flufenamide, or a salt thereof, and γ-cyclodextrin or a pharmaceutically acceptable derivative of γ-cyclodextrin. The invention further provides compositions comprising the pharmaceutical preparation of the invention, and kits and uses of the pharmaceutical preparations of the invention.

### FIELD OF THE INVENTION

The present invention relates to novel pharmaceutical preparations containing melphalan flufenamide, or a salt thereof, and their use. The present invention also provides kits and methods for preparing the novel pharmaceutical preparations of the invention.

### BACKGROUND OF THE INVENTION

Melphalan flufenamide (also known as melflufen, L-Melphalanyl-4-fluoro-L-phenylalanine ethyl ester and ethyl (2S)-2-[[(2S)-2-amino-3-[4-[bis(2-chloroethyl)amino]phenyl]propanoyl]amino]-3-(4-fluoro phenyl)propanoate), is an anti-tumour agent useful in the treatment of cancer, and particularly the treatment of multiple myeloma. Melphalan flufenamide is described in WO 01/96367 and WO 2014/065751.

In February 2021, the United States Food and Drug Administration (FDA) approved the use of the hydrochloride salt of melphalan flufenamide in the treatment of adult patients with relapsed or refractory multiple myeloma. In August 2022 the European Medicines Agency granted a marketing authorisation for the European Union and countries in the European Economic Area (EEA) for the hydrochloride salt of melphalan flufenamide, in combination with dexamethasone, for the treatment of adult patients with multiple myeloma who have received at least three prior lines of therapies, whose disease is refractory to at least one proteasome inhibitor, one immunomodulatory agent, and one anti-CD38 monoclonal antibody, and who have demonstrated disease progression on or after the last therapy. Melphalan flufenamide hydrochloride is sold in the EEA under the trade name PEPAXTI®. It has been sold in the US under the trade name PEPAXTO®. The structure of melphalan flufenamide hydrochloride is shown below:

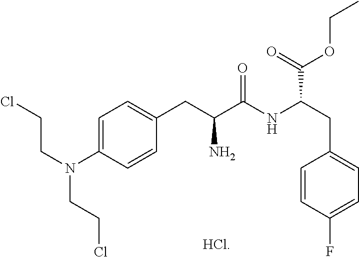 [SMILES: CCOC(=O)[C@H](Cc1ccc(F)cc1)NC(=O)[C@@H](N)Cc1ccc(N(CCCl)CCCl)cc1 ; "HCl."]

PEPAXTI© is a white to off-white lyophilized powder. A single vial of PEPAXTI® contains 20 mg melphalan flufenamide (21.48 mg of melphalan flufenamide hydrochloride) and 1,000 mg sucrose. PEPAXTO® has an identical formulation.

Further lyophilised preparations of melphalan flufenamide are described in WO2012/146625 and WO 2014/065751. Liquid preparations of melphalan flufenamide containing propylene glycol and/or polyethylene glycol as solvents are described in WO2020/212594.

Before administration of PEPAXTI® (or PEPAXTO®), the lyophilized powder must be reconstituted with a 5% glucose solution (w/v in water) and then further diluted to the required concentration with a 0.9% sodium chloride solution (w/v in water) suitable for administration to a patient by intravenous infusion. However, like many alkylating drugs, melphalan flufenamide is unstable in aqueous solution. As such, to ensure that a patient receives a safe and accurate dose of melphalan flufenamide, it is necessary to prepare the infusion solution quickly and to begin infusion to a patient shortly after the reconstitution step.

Improvements to the stability and/or handling properties of melphalan flufenamide preparations, particularly in aqueous solutions, would be advantageous in the clinic and allow, for example, greater flexibility in the storage conditions of melphalan flufenamide and the procedures used for the preparation and administration of injection and infusion solutions.

### SUMMARY OF THE INVENTION

The present invention provides a pharmaceutical preparation comprising melphalan flufenamide, or a salt thereof, and γ-cyclodextrin (γCD) or a pharmaceutically acceptable derivative of γ-cyclodextrin.

The present inventors have found that melphalan flufenamide is surprisingly stable and soluble in aqueous solutions containing γCD. As described herein, in a stability study using melphalan flufenamide hydrochloride (Example compound 1, Ex 1), the present inventors found that following storage of a solution comprising melphalan flufenamide and γCD at 25° C. to 30° C. for 22 hours, the melphalan flufenamide content of the solution decreased by only 31% relative to the melphalan flufenamide content before storage of the solution (i.e. at t₀) at 25° C. to 30° C. In the absence of any excipient, melphalan flufenamide hydrochloride is known to degrade in aqueous solutions at a rate of around 8% to 10% per hour when stored at room temperature. A deuterated analogue of melphalan flufenamide (Example compound 2, Ex 2) was also found to display improved stability in the presence of γCD.

In a further stability study, the present inventors found that the melflufen flufenamide content in a saline infusion solution containing melflufen flufenamide and γCD decreased by about 1% over 24 hours at 5° C. and about 2% over 48 hours at 5° C., whereas the melflufen flufenamide content in a saline infusion solution not comprising γCD decreased by about 5% over 24 hours at 5° C. and about 8% over 48 hours at 5° C. (see FIG. 3). That is to say that melflufen flufenamide displayed a degradation rate of about 1% per 24 hours at 5° C. in a saline infusion solution comprising γCD, and a degradation rate of about 4 to 5% per 24 hours at 5° C. in a saline infusion solution not comprising γCD.

The present inventors have also found that the positive effects of γCD can be obtained with a pharmaceutically acceptable derivative of γ-cyclodextrin. In a further stability study, the present inventors found that the melflufen flufenamide content in a saline infusion solution containing melflufen flufenamide and hydroxypropyl-γCD decreased on average by about 4% over 5 hours at room temperature, whereas the melflufen flufenamide content in a saline infusion solution not comprising any HPγCD decreased on average by about 40% over the same time period and under the same conditions. Similar results were found in a glucose infusion solution. Increased stability was also seen for liquid formulations of melflufen flufenamide and HPγCD in propylene glycol compared to the same liquid formulation not comprising any HPγCD.

As well as improved stability, pharmaceutical preparations of melphalan flufenamide with HPγCD allow for higher reconstitution concentrations of up to 3.0 mg/mL, compared to the PEPTAXI® approved drug product, which has a limited reconstitution concentration of 0.5 mg/mL. As higher concentrations of melphalan flufenamide can be achieved with pharmaceutical preparations of the present invention, less liquid is required for initial reconstitution. This has the advantage that a solid drug product pharmaceutical preparation, such as a lyophilised pharmaceutical preparation, of melphalan flufenamide (or a salt thereof) and a γCD or pharmaceutically acceptable γCD derivative (such as HPγCD), can be provided in a smaller vial.

Finally, the inventors have found that the improved stability of melphalan flufenamine is achieved regardless of how the γCD, or the pharmaceutically acceptable derivative of γCD such as HPγCD, is formulated with the melphalan flufenamine: the melphalan flufenamine can be lyophilised with the γCD, or the pharmaceutically acceptable derivative of γCD such as HPγCD; it can be provided separately and mixed with the melphalan flufenamide on initial reconstitution (i.e. provided in a separate vial to the melphalan flufenamide and mixed with it on initial reconstitution to provide a 0.5 to 3.0 mg/mL solution before further dilution to an infusion concentration of around 0.05 or 0.10 mg/mL), or it can be added to or provided in the infusion solution (for example a physiologically acceptable solution having a concentration of 0.10 to 0.16 mg/mL melphalan flufenamide that can be directly administered to a patient).

Typically, for an infusion solution of melflufen flufenamide to be deemed suitable for administering to a patient, when the solution is to be administered to the patient the melflufen flufenamide content in the solution must be above 90% relative to the intended melflufen flufenamide content when the solution was prepared. Thus, the decreased degradation rate of melflufen flufenamide in the presence of γCD or a pharmaceutically acceptable derivative of γCD allows for longer storage of an infusion solution before administering to a patient and also ensures that the quality of the infusion solution does not decrease substantially during the infusion process.

Furthermore, stabilised aqueous solutions of melphalan flufenamide are especially useful in the clinic as they allow greater flexibility and robustness in the drug preparation, storage and administration protocols. For example, by increasing the stability of melphalan flufenamide in aqueous infusion solutions, there is a dramatic reduction on the burden placed on clinical staff because it allows for more flexibility in the time spent preparing the drug for administration, and more flexibility regarding when the drug can be administered. In addition, the ability to store aqueous solutions of melphalan flufenamide for longer periods allows for the preparation of larger batches of aqueous infusion solutions, which may then be shipped to multiple clinical facilities for administration to patients and/or it allows the staggering of patient appointments over a longer period of time without needing to make new batches of aqueous infusion solutions for patient groups arriving at different times. Such features are especially important for highly potent cytotoxic agents, such as melphalan flufenamide, because they reduce the exposure to clinical staff to the cytotoxic agent (i.e. because batches need to be prepared less often).

Melflufen flufenamide was also found to display a favourable dissolution rate in saline solutions containing γCD or a pharmaceutically acceptable derivative of γCD.

This is particularly beneficial in the clinic because it allows for solutions to be prepared quickly and removes the need for further solvents or diluents to improve the dissolution rate of melflufen flufenamide into an infusion solution.

The present invention also provides a composition comprising a pharmaceutical preparation of the invention and a physiologically acceptable aqueous solvent.

The present invention also provides a kit comprising melphalan flufenamide and γCD or a pharmaceutically acceptable derivative of γ-cyclodextrin, and optionally a physiologically acceptable aqueous solvent.

The present invention also provides a pharmaceutical preparation or composition of the invention for use as a medicament.

The present invention also provides a pharmaceutical preparation or composition of the invention for use in the treatment or prophylaxis of a disease or condition that may be treated with conventional chemotherapy agents, for example, with an alkylator (e.g. melphalan, cyclophosphamide and bendamustine).

The present invention further provides a pharmaceutical preparation or composition of the invention for use in the treatment or prophylaxis of cancer, for example a cancer selected from the list consisting of multiple myeloma, breast cancer, lung cancer, ovarian cancer, glioblastoma, leukaemias and lymphomas.

The present invention further provides a pharmaceutical preparation or composition of the invention for use in the treatment or prophylaxis of amyloidosis.

The present invention also provides a method for treating a patient which comprises administering a pharmaceutically effective amount of a pharmaceutical preparation or composition of the invention to a subject in need thereof.

### DESCRIPTION OF THE DRAWINGS

FIG. 1 shows the change in Example compound 2 (Ex 2) content in a saline solution containing Ex 2 and γCD (top line in the graph), and a glucose solution containing Ex 2 and γCD (second line from the top of the graph) over 18 hours at 25° C. to 30° C. Also shown is the change in Ex 2 content in saline and glucose solutions not containing γCD (third and fourth line from the top of the graph, respectively). For the avoidance of doubt, γCD is referred to in FIG. 1 as “gammaCD”, and melphalan flufenamide is referred to as “melflufen”.

FIG. 2 shows the change in melphalan flufenamide content in a glucose solution containing melphalan flufenamide and γCD over 18 hours at room temperature. The melphalan flufenamide solutions used in the experiment were prepared using a lyophilised melphalan flufenamide preparation containing melphalan flufenamide and sucrose. For the avoidance of doubt, γCD is referred to in FIG. 2 as “gCD” and “gammaCD”, and melphalan flufenamide is referred to as “melflufen”.

FIG. 3 shows the change in melphalan flufenamide content in melphalan flufenamide infusion solutions over 43 hours at 5° C. The infusions solutions were prepared according to the guidance set out in the U.S Prescribing Information for the melphalan flufenamide preparation sold under name PEPTAXTO® dated February 2021. The upper line in the graph shows the change in melphalan flufenamide content in an infusion solution containing γCD (at a weight ratio (w/w) of γCD to melphalan flufenamide of 16:1), and the lower line in the graph shows the change in melphalan flufenamide content in an infusion solution not containing γCD. For the avoidance of doubt, γCD is referred to in FIG. 3 as “gCD”, and melphalan flufenamide is referred to as “melflufen”.

### DETAILED DESCRIPTION

The present invention provides pharmaceutical preparations comprising melflufen flufenamide, or salts thereof, and γ-cyclodextrin (γCD) or a pharmaceutically acceptable derivative of γ-cyclodextrin. As described in the Examples section, the pharmaceutical preparations of the present invention display excellent stability in aqueous solutions.

### Melphalan Flufenamide

For the avoidance of doubt, in this document, when the term “melphalan flufenamide” is used, it is also intended to include salt(s) thereof, even if this is not explicitly stated.

Also for the avoidance of doubt, in this document, when the term “melphalan flufenamide” is used, it includes isotopic derivatives of melphalan flufenamide, unless stated otherwise. Particular examples of isotopic derivatives of melphalan flufenamide suitable for use in the present invention are deuterated melphalan flufenamide derivatives. Examples of deuterated melflufen derivatives, and methods of making deuterated melphalan flufenamide derivatives, are described in WO2020/079165, the content of which is incorporated herein by reference.

In certain embodiments, a deuterated melphalan flufenamide derivative suitable for use in the present invention is (²H₅)ethyl (2S)-2-[(2S)-2-amino-3-{4-[bis(2-chloroethyl)amino]phenyl}propanamido]-3-(4-fluorophenyl)propanoate hydrochloride (hereinafter referred to as Ex 2). Ex 2 has the following structure:

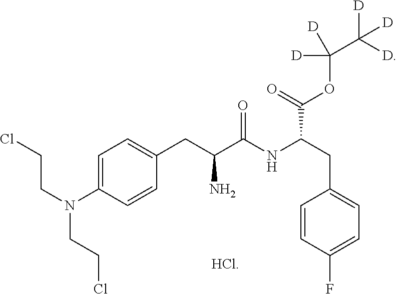

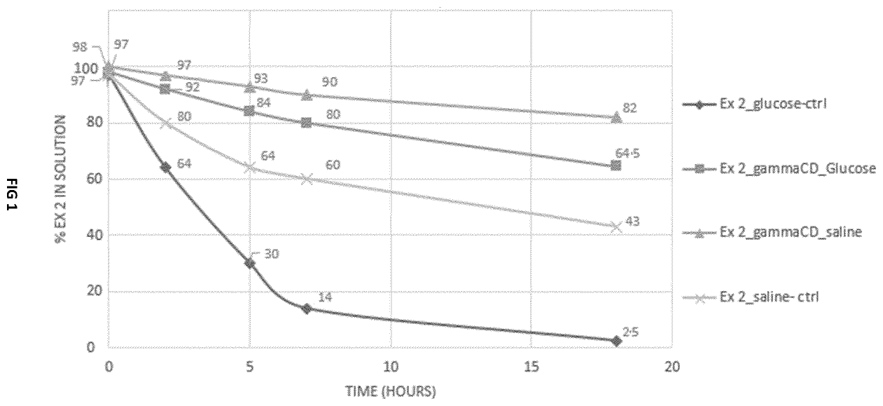

The present inventors have found that the pharmaceutical preparations of the present invention are particularly suitable for isotopic derivatives of melphalan flufenamide such as Ex 2 (see Example 3 and FIG. 1 of the present application). Thus, in one preferred embodiment, the pharmaceutical preparation of the invention comprises Ex 2, or a salt thereof, and γCD or a pharmaceutically acceptable derivative of γ-cyclodextrin (such as HPγCD). In another preferred embodiment, the pharmaceutical preparation of the invention comprises Ex 2, or a salt thereof, and γCD. The present inventors have also found that the pharmaceutical preparations of the present invention are particularly suitable for melphalan flufenamide with a natural isotope abundance. Thus, in another preferred embodiment, the pharmaceutical preparation of the invention comprises melphalan flufenamide with a natural isotope abundance, or a salt thereof, and γCD or a pharmaceutically acceptable derivative of γ-cyclodextrin (such as HPγCD).

Also, for the avoidance of doubt, when referred to in this document, the mass of melphalan flufenamide is the mass of the melphalan flufenamide molecule excluding the mass of any counterion (i.e. melphalan flufenamide as it free base) unless explicitly stated otherwise. The molecular weight of counterion-free melflufen (also referred to herein as the “free base”) (with a natural isotope abundance) is 498.42 g/mol. Melphalan flufenamide, and salts thereof, especially the hydrochloride salt thereof, are known from, for example, WO 01/96367, WO 2014/065751 and WO 2020/212594, and the same salts are suitable for use in the present invention. Salts of melphalan flufenamide which are suitable for use in the present invention are those wherein a counterion is pharmaceutically acceptable. Suitable salts include those formed with organic or inorganic acids. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄) alkyl or aryl sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxalic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine.

Preferred salts of melphalan flufenamide include acid addition salts such as those formed from hydrochloric, hydrobromic, acetic, p-toluenesulfonic, tartaric, sulphuric, succinic, phosphoric, oxalic, nitric, methanesulfonic, malic, maleic and citric acid. Typically, the salt of melphalan flufenamide for use according to the present invention is the hydrochloride salt (i.e. the addition salt formed from hydrochloric acid).

Other preferred salts of melphalan flufenamide include hydrochloric, hydrobromic, nitric, methansulphonic, sulphuric, phosphoric, trifluoroacetic, para-toluene sulphonic, 2-mesitylen sulphonic, citric, acetic, tartaric, fumaric, lactic, succinic, malic, malonic, maleic, 1,2-ethanedisulphonic, adipic, aspartic, benzenesulphonic, benzoic, ethanesulphonic or nicotinic acid.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. The complex may incorporate a solvent in stoichiometric or non-stoichiometric amounts. Solvates are described in Water-Insoluble Drug Formulation, 2^{nd} ed R. Lui CRC Press, page 553 and Byrn et al Pharm Res 12(7), 1995, 945-954. Before it is made up in solution, the melphalan flufenamide, or salt thereof, for use in the present invention may be in the form of a solvate. Solvates of melphalan flufenamide that are suitable for use according to the present invention are those wherein the associated solvent is pharmaceutically acceptable. For example a hydrate is a pharmaceutically acceptable solvate.

### Pharmaceutical Preparations

The present invention provides pharmaceutical preparations comprising melphalan flufenamide, or a salt thereof, and γCD or a pharmaceutically acceptable derivative of γ-cyclodextrin.

γCD is a cyclic oligosaccharide composed of eight α-1,4-linked glucopyranose units. It is a known excipient for pharmaceutical and nutritional products, and has been used as a delivery vehicle for hydrophobic or poorly water-soluble molecules.

Various pharmaceutically acceptable derivatives of γ-cyclodextrin are known. Preferably, such derivatives are soluble in water, for example to a concentration of at least 5 wt % at room temperature (i.e. at least 50 g/L), for example at least 10 wt %. Examples of a pharmaceutically acceptable derivative of γ-cyclodextrin include, but are not limited to, hydroxypropyl-γ-cyclodextrin, succinyl-γ-cyclodextrin, sulfobutyl ether-γ-cyclodextrin, γ-cyclodextrin phosphate sodium salt, γ-cyclodextrin sulphate sodium salt, 2-carboxyethyl-γ-cyclodextrin, acetylated-γ-cyclodextrin, and sugammadex. For example, hydroxypropyl-γCD (HPγCD) is highly suitable for use in pharmaceutical preparations, especially in pharmaceutical preparations, formulation and compositions of the present invention. HPγCD is an especially preferred pharmaceutically acceptable derivative of γCD according to the present invention. In this specification, unless the context requires otherwise, comments regarding γCD apply equally to derivatives of γCD (and in particular pharmaceutically acceptable derivatives of γCD; for example, HPγCD, succinyl-γ-cyclodextrin, sulfobutyl ether-γ-cyclodextrin, γ-cyclodextrin phosphate sodium salt, γ-cyclodextrin sulphate sodium salt, 2-carboxyethyl-γ-cyclodextrin, acetylated-γ-cyclodextrin, and sugammadex; and in particular HPγCD).

For the avoidance of doubt, in embodiments where a pharmaceutical preparation of the present invention comprises a pharmaceutically acceptable derivative of γ-cyclodextrin, the pharmaceutically acceptable derivative of γ-cyclodextrin may have any Degree of Substitution (DS; also known as Degree of Molar Substitution). DS as used herein is the average number of derivative group substituents (for example, the average number of hydroxypropyl, sulfobutyl ether, 2-carboxyethyl, acetyl and/or 3-sulfanylpropanoic acid substituents) that have reacted with one glucopyranose repeat unit of the γCD. Each glucopyranose repeat unit has 3 hydroxyl groups that can be substituted by derivative group substituents. The DS number represents the average DS of a mixture of isomers. It is noted that for some derivative group substituents (such as hydroxypropyl groups), the electrophile used to react with γCD to form the derivative of γCD (e.g. propylene oxide) can react with hydroxyl groups of the derivative group substituent itself, as well as those of the glucopyranose repeat unit, forming a polymeric side chain (e.g. polypropylene glycol). Thus, the DS value for a derivative of γ-cyclodextrin can be greater than 3 when two or more substituents react to form oligomeric or polymeric side chains.

As an illustrative example, a pharmaceutically acceptable derivative of γ-cyclodextrin of the present invention (e.g. HPγCD) having a DS of about 0.6 would have, on average, 0.6 derivative group substituents (e.g. hydroxypropyl groups) reacted with one glucopyranose repeat unit of the γCD. As γCD has 8 glucopyranose repeat units, a pharmaceutically acceptable derivative of γ-cyclodextrin defined as having a DS of 0.6 would have, on average, about 4.7 (for example 4.7 or 4.8) derivative group substituents (e.g. hydroxypropyl groups) per γCD molecule.

In certain embodiments, a pharmaceutical preparation of the present invention comprises a pharmaceutically acceptable derivative of γ-cyclodextrin having a DS of at least 0.04, for example a DS of about 0.04 to about 5; for example about 0.04 to about 4, about 0.04 to about 3, or about 0.04 to about 3. In certain embodiments, a pharmaceutical preparation of the present invention comprises a pharmaceutically acceptable derivative of γ-cyclodextrin having a DS of at least 0.08, 0.1, at least 0.2 at least 0.4 or at least 0.5. In certain embodiments, a pharmaceutical preparation of the present invention comprises a pharmaceutically acceptable derivative of γ-cyclodextrin having a DS of less than 5, less than 4, less than 3, less than 2, less than 1.5, less than 1 or less than 0.8. In certain embodiments, a pharmaceutically acceptable derivative of γ-cyclodextrin of the present invention has a DS of about 0.04 to about 2, for example about 0.1 to about 1.5, about 0.1 to about 1, about 0.2 to about 1, about 0.2 to about 0.8, or about 0.4 to about 0.8. In one preferred embodiment, the pharmaceutically acceptable derivative of γ-cyclodextrin is HPγCD having a DS of about 0.04 to about 1, more preferably about 0.1 to about 1, more preferably about 0.2 to about 0.8, and most preferably 0.4 to 0.8, for example 0.4, 0.5, 0.6, 0.7 or 0.8. In one preferred embodiment the pharmaceutically acceptable derivative of γ-cyclodextrin is HPγCD having a DS of about 0.6.

The pharmaceutical preparations of the present invention comprise γCD (or a pharmaceutically acceptable derivative of γ-cyclodextrin, such as HPγCD) at a weight ratio (w/w) of γCD (or a pharmaceutically acceptable derivative of γ-cyclodextrin, such as HPγCD) to melphalan flufenamide of about 1:1 to about 100:1. Typically, the pharmaceutical preparation comprises γCD (or a pharmaceutically acceptable derivative of γ-cyclodextrin, such as HPγCD) at a weight ratio (w/w) of γCD (or a pharmaceutically acceptable derivative of γ-cyclodextrin, such as HPγCD) to melphalan flufenamide of about 4:1 to about 80:1.

Melphalan flufenamide displays especially good stability in aqueous solutions when γCD (or a pharmaceutically acceptable derivative of γ-cyclodextrin, such as HPγCD) is included in the pharmaceutical preparation at a weight ratio (w/w) of γCD (or a pharmaceutically acceptable derivative of γ-cyclodextrin, such as HPγCD) to melphalan flufenamide of about 4:1 to about 25:1. For example, the present inventors have demonstrated that an aqueous solution of melphalan flufenamide containing γCD at a weight ratio (w/w) of γCD to melphalan flufenamide of about 4:1 retained 69% of the melphalan flufenamide content after 22 hours at 25° C. to 30° C.

In further stability studies, the present inventors have demonstrated that melphalan flufenamide displays especially good stability in aqueous solutions containing γCD (or a pharmaceutically acceptable derivative of γ-cyclodextrin, such as HPγCD) at a weight ratio (w/w) of γCD (or a pharmaceutically acceptable derivative of γ-cyclodextrin, such as HPγCD) to melphalan flufenamide of about 10:1 to about 50:1, about 10:1 to about 30:1, about 10:1 to about 28:1, about 10:1 to about 25:1 and about 10:1 to about 23:1.

In further stability studies, the present inventors have demonstrated that melphalan flufenamide displays especially good stability in aqueous solutions containing γCD (or a pharmaceutically acceptable derivative of γ-cyclodextrin, such as HPγCD) at a weight ratio (w/w) of γCD (or a pharmaceutically acceptable derivative of γ-cyclodextrin, such as HPγCD) to melphalan flufenamide of about 10:1 to about 50:1, about 10:1 to about 30:1, about 10:1 to about 28:1, about 10:1 to about 25:1 and about 10:1 to about 23:1. In certain stability studies, the present inventors have demonstrated that melphalan flufenamide displays especially good stability in aqueous solutions containing γCD (or a pharmaceutically acceptable derivative of γ-cyclodextrin, such as HPγCD) at a weight ratio (w/w) of γCD (or a pharmaceutically acceptable derivative of γ-cyclodextrin, such as HPγCD) to melphalan flufenamide of about 15:1 to about 50:1, about 15:1 to about 30:1, about 15:1 to about 28:1, and about 15:1 to about 25:1.

Thus, preferably, the pharmaceutical preparations of the present invention comprise γCD (or a pharmaceutically acceptable derivative of γ-cyclodextrin, such as HPγCD) at a weight ratio (w/w) of γCD (or a pharmaceutically acceptable derivative of γ-cyclodextrin, such as HPγCD) to melphalan flufenamide of about 4:1 to about 25:1. In exemplary embodiments, the pharmaceutical preparations of the present invention may comprise γCD (or a pharmaceutically acceptable derivative of γ-cyclodextrin, such as HPγCD) at a weight ratio (w/w) of γCD (or a pharmaceutically acceptable derivative of γ-cyclodextrin, such as HPγCD) to melphalan flufenamide of at least about 5:1. In certain preferred embodiments, the pharmaceutical preparations of the present invention comprise γCD (or a pharmaceutically acceptable derivative of γ-cyclodextrin, such as HPγCD) at a weight ratio (w/w) of γCD (or a pharmaceutically acceptable derivative of γ-cyclodextrin, such as HPγCD) to melphalan flufenamide of about 5:1 to about 25:1. More preferably, the weight ratio (w/w) of γCD (or a pharmaceutically acceptable derivative of γ-cyclodextrin, such as HPγCD) to melphalan flufenamide is about 5:1 to about 25:1, for example about 10:1 to about 25:1 (for example, the pharmaceutical preparations of the present invention may comprise γCD at a weight ratio (w/w) of γCD to melphalan flufenamide of about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, or about 25:1). In one preferred embodiment, the weight ratio (w/w) of γCD (or a pharmaceutically acceptable derivative of γ-cyclodextrin, such as HPγCD) to melphalan flufenamide is about 10:1 to about 23:1 (for example, the weight ratio (w/w) of γCD (or a pharmaceutically acceptable derivative of γ-cyclodextrin, such as HPγCD) to melphalan flufenamide is about 10:1, about 15:1, about 18:1, or about 23:1).

In exemplary embodiments, wherein the pharmaceutical preparation of the present invention comprises γCD at a weight ratio (w/w) of γCD (or a pharmaceutically acceptable derivative of γ-cyclodextrin, such as HPγCD) to melphalan flufenamide of about 5:1 to about 25:1, the pharmaceutical preparation may comprise, for example, 1 mg to 30 mg melphalan flufenamide (excluding the mass of any counterion) and about 5 mg to about 750 mg γCD (or a pharmaceutically acceptable derivative of γ-cyclodextrin, such as HPγCD). For example, a pharmaceutical preparation comprising 20 mg melphalan flufenamide (excluding the mass of any counterion) may comprise about 100 mg to about 500 mg γCD (for example, about 200 mg to about 500 mg γCD(or a pharmaceutically acceptable derivative of γ-cyclodextrin, such as HPγCD)).

In another exemplary embodiment, wherein the pharmaceutical preparation of the present invention comprises γCD at a weight ratio (w/w) of γCD (or a pharmaceutically acceptable derivative of γ-cyclodextrin, such as HPγCD) to melphalan flufenamide of about 15:1 to about 25:1, the pharmaceutical preparation may comprise, for example, 1 mg to 30 mg melphalan flufenamide (excluding the mass of any counterion) and about 15 mg to about 750 mg γCD (or a pharmaceutically acceptable derivative of γ-cyclodextrin, such as HPγCD). For example, a pharmaceutical preparation comprising 20 mg melphalan flufenamide (excluding the mass of any counterion) may comprise about 300 mg to about 500 mg γCD (for example, about 320 mg to about 500 mg γCD (or a pharmaceutically acceptable derivative of γ-cyclodextrin, such as HPγCD)).

In another exemplary embodiment, wherein the pharmaceutical preparation of the present invention comprises γCD at a weight ratio (w/w) of γCD (or a pharmaceutically acceptable derivative of γ-cyclodextrin, such as HPγCD) to melphalan flufenamide of about 20:1 to about 25:1, the pharmaceutical preparation may comprise, for example, 1 mg to 30 mg melphalan flufenamide (excluding the mass of any counterion) and about 20 mg to about 750 mg γCD (or a pharmaceutically acceptable derivative of γ-cyclodextrin, such as HPγCD). For example, a pharmaceutical preparation comprising 20 mg melphalan flufenamide (excluding the mass of any counterion) may comprise about 400 mg to about 500 mg γCD (for example, about 400 mg, about 420 mg, about 440 mg, about 460 mg, about 480 mg, or about 500 mg γCD (or a pharmaceutically acceptable derivative of γ-cyclodextrin, such as HPγCD)).

Melphalan flufenamide displays especially good stability in aqueous solutions when HPγCD is included in the pharmaceutical preparation at a weight ratio (w/w) of HPγCD to melphalan flufenamide of about 10:1 to about 50:1, for example about 20:1 to about 40:1 or about 15:1 to about 25:1. For example, the present inventors have demonstrated that an aqueous solution of melphalan flufenamide containing HPγCD at a weight ratio (w/w) of HPγCD to melphalan flufenamide of about 36:1 retained 87% of the melphalan flufenamide content after 18 hours at room temperature. As a further example, the present inventors have demonstrated that an aqueous solution of 0.2 mg/mL melphalan flufenamide containing HPγCD at a weight ratio (w/w) of HPγCD to melphalan flufenamide of 1:20, 1:24, 1:28, 1:32, 1:36 or 1:40 retained over 78% of the melphalan flufenamide content after 18 hours at room temperature, and over 93% of the melphalan flufenamide content after 18 hours in a fridge. As another example, the present inventors have demonstrated that an aqueous solution of 0.1 and 0.5 mg/mL melphalan flufenamide containing HPγCD at a weight ratio (w/w) of HPγCD to melphalan flufenamide of 1:16, 1:20, 1:24, or 1:28 retained over 80% and 90%, respectively, of the melphalan flufenamide content after 20 hours at room temperature.

Therefore, in exemplary embodiments, wherein the pharmaceutical preparation of the present invention comprises HPγCD at a weight ratio (w/w) of HPγCD to melphalan flufenamide of about 15:1 to about 40:1, the pharmaceutical preparation may comprise, for example, 1 mg to 30 mg melphalan flufenamide (excluding the mass of any counterion) and about 15 mg to about 1200 mg HPγCD. For example, a pharmaceutical preparation comprising 20 mg melphalan flufenamide (excluding the mass of any counterion) may comprise about 300 mg to about 800 mg HPγCD (for example, about 500 mg to about 700 mg HPγCD).

In exemplary embodiments, wherein the pharmaceutical preparation of the present invention comprises HPγCD at a weight ratio (w/w) of HPγCD to melphalan flufenamide of about 20:1 to about 40:1, the pharmaceutical preparation may comprise, for example, 1 mg to 30 mg melphalan flufenamide (excluding the mass of any counterion) and about 20 mg to about 1200 mg HPγCD. For example, a pharmaceutical preparation comprising 20 mg melphalan flufenamide (excluding the mass of any counterion) may comprise about 400 mg to about 800 mg HPγCD (for example, about 500 mg to about 700 mg HPγCD). In further exemplary embodiments, wherein the pharmaceutical preparation of the present invention comprises HPγCD at a weight ratio (w/w) of HPγCD to melphalan flufenamide of about 32:1 to about 40:1, the pharmaceutical preparation may comprise, for example, 1 mg to 30 mg melphalan flufenamide (excluding the mass of any counterion) and about 32 mg to about 1200 mg HPγCD. For example, a pharmaceutical preparation comprising 20 mg melphalan flufenamide (excluding the mass of any counterion) may comprise about 640 mg to about 800 mg HPγCD (for example, about 680 mg to about 760 mg HPγCD).

In a further preferred exemplary embodiment, wherein the pharmaceutical preparation of the present invention comprises HPγCD at a weight ratio (w/w) of HPγCD to melphalan flufenamide of about 15:1 to about 25:1, the pharmaceutical preparation may comprise, for example, 1 mg to 30 mg melphalan flufenamide (excluding the mass of any counterion) and about 15 mg to about 750 mg HPγCD. For example, a pharmaceutical preparation comprising 20 mg melphalan flufenamide (excluding the mass of any counterion) may comprise about 300 mg to about 500 mg HPγCD (for example, about 300 mg, about 320 mg, about 360 mg, about 400 mg, about 440 mg, about 480 mg, or about 500 mg HPγCD).

In a further preferred exemplary embodiment, wherein the pharmaceutical preparation of the present invention comprises HPγCD at a weight ratio (w/w) of HPγCD to melphalan flufenamide of about 20:1 to about 25:1, the pharmaceutical preparation may comprise, for example, 1 mg to 30 mg melphalan flufenamide (excluding the mass of any counterion) and about 20 mg to about 750 mg HPγCD. For example, a pharmaceutical preparation comprising 20 mg melphalan flufenamide (excluding the mass of any counterion) may comprise about 400 mg to about 500 mg HPγCD (for example, about 400 mg, about 420 mg, about 440 mg, about 460 mg, about 480 mg, or about 500 mg HPγCD).

Additional excipients may be included in pharmaceutical preparations of the present invention. A further excipient may be included in the pharmaceutical preparation, for example, to improve the dissolution rate of melphalan flufenamide into an aqueous solution and/or improve the stability of melphalan flufenamide when in its lyophilised form. Notably, the present inventors have found that γCD, and pharmaceutically acceptable derivatives of γCD (such as HPγCD), have a stabilising effect on melphalan flufenamide even when further excipients are included in the pharmaceutical preparation. Thus, in certain embodiments, the pharmaceutical preparations of the present invention comprise one or more further pharmaceutically acceptable excipients, in addition to the γCD or the pharmaceutically acceptable derivative of γ-cyclodextrin (such as HPγCD).

The one or more further pharmaceutically acceptable excipients may be selected from the group consisting of polysorbate 80, propylene glycol, sucrose, trehalose dehydrate, MgCl₂, and CaCl₂. Preferably, the further pharmaceutically acceptable excipient is sucrose.

Typically, when the pharmaceutical preparation of the present invention comprises one or more further excipients in addition to γCD, the one or more further excipients are each independently present in the preparation at a weight ratio (w/w) of excipient to melphalan flufenamide of about 1:1 to about 100:1. For example, about 1:1 to about 50:1.

In certain embodiments, the pharmaceutical preparation of the present invention is a lyophilized powder. The present inventors have found that γCD. or pharmaceutically acceptable derivatives of γ-cyclodextrin (such as HPγCD), can be included in a lyophilised melphalan flufenamide formulation. Reconstitution of the lyophilised powder provides an aqueous solution in which melphalan flufenamide is especially stable.

A pharmaceutical preparation of the present invention, when in the form of a lyophilised powder, may essentially consist of melphalan flufenamide and γCD or a pharmaceutically acceptable derivative of γ-cyclodextrin (such as HPγCD). Alternatively, a pharmaceutical preparation of the present invention, when in the form of a lyophilised powder, comprises one or more further excipient in addition to the γCD or the pharmaceutically acceptable derivative of γ-cyclodextrin (such as HPγCD), and in particular one further excipient in addition to the γCD or the pharmaceutically acceptable derivative of γ-cyclodextrin (such as HPγCD). A further excipient may be selected from the group consisting of sucrose, polysorbate 80, propylene glycol, trehalose dehydrate, MgCl₂, and CaCl₂. Preferably, the further excipient is sucrose.

In exemplary embodiments, wherein the pharmaceutical preparation is a lyophilised powder, the pharmaceutical preparation comprises (or consists essentially of) melphalan flufenamide, γCD or a pharmaceutically acceptable derivative of γ-cyclodextrin (such as HPγCD), and sucrose. Sucrose has been found to improve the dissolution rate of melphalan flufenamide into aqueous solutions. When sucrose is present in a pharmaceutical preparation of the present invention, it is typically present at a weight ratio (w/w) of sucrose to melphalan flufenamide of about 1:1 to about 500:1. For example, the sucrose may be present at a weight ratio (w/w) of sucrose to melphalan flufenamide of about 2:1 to about 100:1, about 1:2 to about 1:75, or about 1:4 to about 1:75. Preferably, the sucrose is present at a weight ratio (w/w) of sucrose to melphalan flufenamide of about 20:1 to about 75:1. More preferably, the sucrose is present at a weight ratio (w/w) of sucrose to melphalan flufenamide of about 25:1 to about 75:1. Even more preferably, the sucrose is present at a weight ratio (w/w) of sucrose to melphalan flufenamide of about 30:1 to about 70:1, about 40:1 to about 60:1, or about 45:1 to about 55:1 (for example 50:1).

In certain embodiments wherein the pharmaceutical preparation is a lyophilized powder, the weight ratio (w/w) of γCD or a pharmaceutically acceptable derivative of γ-cyclodextrin (such as HPγCD) to melphalan flufenamide is about 4:1 to about 80:1 (for example, about 5:1 to about 25:1), and the weight ratio (w/w) of sucrose to melphalan flufenamide is about 25:1 to 75:1 (for example, 50:1). For example, the weight ratio (w/w) of γCD or a pharmaceutically acceptable derivative of γ-cyclodextrin (such as HPγCD) to melphalan flufenamide is about 10:1 to about 25:1, and the weight ratio (w/w) of sucrose to melphalan flufenamide is about 25:1 to 75:1 (for example, 50:1). An exemplary embodiment of such a preparation comprises 1 mg to 30 mg melphalan flufenamide (excluding the mass of any counterion), about 5 mg to about 750 mg γCD or a pharmaceutically acceptable derivative of γ-cyclodextrin (such as HPγCD) and about 0.25 g to 2.25 g of sucrose. In a further exemplary embodiment, such a preparation may comprise 20 mg of melphalan flufenamide (excluding the mass of any counterion), 1 g of sucrose, and about 200 mg to about 500 mg γCD or a pharmaceutically acceptable derivative of γ-cyclodextrin (such as HPγCD) (for example, 200 mg, 250 mg, 300 mg, 350 mg, 400 mg, 450 mg or 500 mg of γCD or a pharmaceutically acceptable derivative of γ-cyclodextrin (such as HPγCD)).

Preferably, in embodiments wherein the pharmaceutical preparation is a lyophilized powder, the pharmaceutical preparation is free, or substantially free, from any organic solvent. By “free” from any organic solvent is it meant that the lyophilized powder pharmaceutical preparation does not contain any measurable amount of an organic solvent. By “substantially free” from any organic solvent it is meant that the lyophilized powder pharmaceutical preparation comprises only trace amounts of an organic solvent, such as less than about a total of about 0.1% w/v of an organic solvent.

In certain embodiments, the pharmaceutical preparation of the present invention is a liquid pharmaceutical formulation. That is to say that the formulation is in the form of a liquid when stored at room temperature.

In embodiments wherein the pharmaceutical preparation is a liquid pharmaceutical formulation, the pharmaceutical preparation comprises (or consists essentially of) melphalan flufenamide, γCD or a pharmaceutically acceptable derivative of γ-cyclodextrin (such as HPγCD) and a pharmaceutically acceptable organic solvent. Typically, the pharmaceutically acceptable organic solvent is selected from the group consisting of propylene glycol, polyethylene glycol (PEG), and mixtures thereof. In one embodiment, the pharmaceutical preparation is a liquid pharmaceutical formulation, and the pharmaceutical preparation comprises (or consists of) melphalan flufenamide, γCD or a pharmaceutically acceptable derivative of γ-cyclodextrin (such as HPγCD), and propylene glycol.

In embodiments wherein the pharmaceutical preparation is a liquid pharmaceutical formulation, the pharmaceutical preparation may optionally contain one or more physiologically acceptable aqueous solvents. Suitable aqueous solvents include non-toxic, parenterally acceptable diluents or solvents, such as water, Ringer's solution, glucose solution and sodium chloride solutions (for example, isotonic sodium chloride solutions). The pharmaceutical preparation may further optionally contain additional components such as anti-oxidants, buffering agents, bacteriostats, and solutes which render the pharmaceutical formulation of the invention isotonic with the blood of the intended recipient.

In an alternative embodiment wherein the pharmaceutical preparation is a liquid pharmaceutical formulation, the pharmaceutical preparation may optionally be substantially free from physiologically acceptable aqueous solvents. In a further alternative embodiment wherein the pharmaceutical preparation is a liquid pharmaceutical formulation, the pharmaceutical preparation may optionally be substantially free from aqueous solvents. By “substantially free” from physiologically acceptable aqueous solvents or aqueous solvents it is meant that the pharmaceutical preparation liquid formulation only comprises trace amounts of physiologically acceptable aqueous solvents or aqueous solvents. For example, less than about 1% w/v physiologically acceptable aqueous solvents or aqueous solvents. Preferably less than 0.5% w/v physiologically acceptable aqueous solvents or aqueous solvents, and more preferably less than 0.1% w/v of physiologically acceptable aqueous solvents or aqueous solvents.

In certain embodiments wherein the pharmaceutical preparation is a liquid pharmaceutical formulation, the pharmaceutical preparation consists essentially of melphalan flufenamide, or a salt thereof, γCD and propylene glycol.

In other embodiments wherein the pharmaceutical preparation is a liquid pharmaceutical formulation, the pharmaceutical preparation consists essentially of melphalan flufenamide, or a salt thereof, γCD and PEG.

In certain embodiments wherein the pharmaceutical preparation is a liquid pharmaceutical formulation, the pharmaceutical preparation consists essentially of melphalan flufenamide, or a salt thereof, HPγCD and propylene glycol.

In other embodiments wherein the pharmaceutical preparation is a liquid pharmaceutical formulation, the pharmaceutical preparation consists essentially of melphalan flufenamide, or a salt thereof, HPγCD and PEG.

A pharmaceutical preparation of the present invention comprising PEG, typically comprises PEG that has an average molecular weight of 150 to 650 Da, for example 150 to 250, 250 to 350, 350 to 450, 450 to 550, and 550 to 650 Da. For example, the PEG may be PEG 200, PEG 300, PEG 400 or PEG 600 as defined in national pharmacopoeias. Preferably, the PEG has an average molecular weight of 250 to 350 Da. A pharmaceutical preparation of the present invention may comprise a mixture of two or more different PEG compounds, for example a mixture comprising two or more of PEG 200, PEG 300, PEG 400 and PEG 600.

In other embodiments, the pharmaceutical preparation comprises melphalan flufenamide, or a salt thereof, γCD or a pharmaceutically acceptable derivative of γ-cyclodextrin (such as HPγCD), propylene glycol and PEG. In certain embodiments, the pharmaceutical preparation consists essentially of melphalan flufenamide, or a salt thereof, γCD or a pharmaceutically acceptable derivative of γ-cyclodextrin (such as HPγCD), propylene glycol and PEG.

For pharmaceutical preparation of the invention containing PEG (for example PEG 200, PEG 300, PEG 400 or PEG 600) and propylene glycol, the weight ratio (w/w) of the propylene glycol to PEG content may be, for example, about 1:1 to about 1:50 (for example, 0.5:1, 1:1, 1:2, 1:5, 1:10, 1:15, 1:20, 1:25, 1:30, 1:35, 1:40, 1:45, 1:50) or about 1:1 to about 50:1 (for example, 1:0.5, 2:1, 5:1, 10:1, 15:1, 20:1, 25:1, 30:1, 35:1, 40:1, 45:1 or 50:1).

In embodiments of the invention wherein the pharmaceutical preparation contains PEG and propylene glycol, the PEG content may be formed from one PEG compound as described above, or formed from a mixture of two or more different PEG compounds, as described above.

Propylene glycol and PEG suitable for use according to the invention are readily available from commercial sources. It should be understood that the source and/or grade of the propylene glycol, PEG and/or any other solvent present in the pharmaceutical preparation of invention are not relevant for the beneficial effects of the present invention to be realised.

In certain embodiments wherein the pharmaceutical preparation is a liquid formulation, the pharmaceutical preparation is substantially free from organic solvents that are not PEG or propylene glycol. By “substantially free” from organic solvents it is meant that the pharmaceutical preparation liquid formulation only comprises trace amounts of an organic solvent, other than propylene glycol and PEG.

For example, less than about 1% w/v of an organic solvent other than propylene glycol or PEG. Preferably less than 0.5% w/v, and more preferably less than 0.1% w/v of an organic solvent other than propylene glycol and PEG. In embodiments wherein the pharmaceutical preparation is a liquid formulation, melphalan flufenamide may be (for example is) present at a concentration of about 1 mg/mL to about 50 mg/mL (excluding the mass of any counterion). Typically, melphalan flufenamide is at concentration of about 10 mg/mL to about 30 mg/mL (excluding the mass of any counterion). For example, 15 mg/mL to 25 mg/mL or 17.5 mg/mL to 22.5 mg/mL (excluding the mass of any counterion). For example, such a liquid formulation may contain melphalan flufenamide at a concentration of 20 mg/mL of melphalan flufenamide (excluding the mass of any counterion) (and, for example, γCD or a pharmaceutically acceptable derivative of γ-cyclodextrin (such as HPγCD) at a concentration of about 200 mg/mL to about 500 mg/mL).

In embodiments wherein the pharmaceutical preparation is a liquid formulation, γCD or a pharmaceutically acceptable derivative of γ-cyclodextrin (such as HPγCD) may be (for example is) present at a concentration of about 1 mg/mL to about 5000 mg/mL (excluding the mass of any counterion). Typically, γCD or a pharmaceutically acceptable derivative of γ-cyclodextrin (such as HPγCD) is at concentration of about 40 mg/mL to about 1200 mg/mL (excluding the mass of any counterion). For example, about 60 to about 1000 mg/mL or about 70 mg/mL to about 900 mg/L (excluding the mass of any counterion). In one embodiment, γCD or a pharmaceutically acceptable derivative of γ-cyclodextrin (such as HPγCD) is at concentration of 100 mg/mL to 500 mg/mL or 150 mg/mL to 600 mg/L (excluding the mass of any counterion). In another embodiment, γCD or a pharmaceutically acceptable derivative of γ-cyclodextrin (such as HPγCD) is at concentration of 200 mg/mL to 500 mg/mL (excluding the mass of any counterion). For example, such a liquid formulation may contain melphalan flufenamide at a concentration of 15 mg/mL to 25 mg/mL or 17.5 mg/mL to 22.5 mg/mL of melphalan flufenamide (excluding the mass of any counterion) and γCD or a pharmaceutically acceptable derivative of γ-cyclodextrin (such as HPγCD) at a concentration of about 200 mg/mL to about 500 mg/mL.

The pharmaceutical preparations of the present invention, when in the form of a liquid pharmaceutical formulation, may be administered directly to a subject, for example without prior dilution, for example, without prior dilution with a physiologically acceptable solvent or diluent. Thus, in one preferred embodiment of the invention, the pharmaceutical preparation is in a form suitable for administering directly to a subject without dilution. In another preferred embodiment, the pharmaceutical preparation is in a form suitable for administering to a patient following dilution, for example following dilution with a physiologically acceptable solvent or diluent, for example a saline solution, a glucose solution, or a mixture thereof.

In certain embodiments wherein the pharmaceutical preparation is a liquid pharmaceutical formulation, the pharmaceutical preparation preferably does not contain any additional physiologically acceptable solvent. Alternatively, or additionally, in certain embodiments wherein the pharmaceutical preparation is a liquid pharmaceutical formulation, the pharmaceutical preparation preferably does not contain any additional physiologically acceptable aqueous solvent.

In certain embodiments wherein the pharmaceutical preparation is a liquid formulation, the pharmaceutical preparation is substantially free from water. By “substantially free” from water it is meant that the pharmaceutical preparation liquid formulation only comprises trace amounts of water. For example, less than about 1% w/v water. Preferably less than 0.5% w/v water, and more preferably less than 0.1% w/v of water.

The present invention also provides a composition comprising a pharmaceutical preparation of the present invention and a physiologically acceptable aqueous solvent or diluent. The compositions of the invention may be suitable for parenteral administration to a subject. As described in the Examples section, injection and infusion solutions may be prepared by diluting a lyophilised powder or a liquid pharmaceutical formulation of the present invention with one or more physiologically acceptable solvent or diluents. Exemplary solvents or diluents include mannitol, glucose, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

In certain embodiments, the pharmaceutical preparation of the invention is administered to the subject following dilution, for example, with a physiologically acceptable aqueous solvent or diluent such as a saline solution, a glucose solution, or a mixture thereof. Any such solvent or diluent may optionally be buffered.

Preferably, the physiologically acceptable solvent is an aqueous solution such as a glucose solution (for example, a 5% glucose solution (w/v in water)), a saline solution (for example, a 0.9% sodium chloride solution (w/v in water)), or a mixture thereof.

A pharmaceutical preparation of the invention may be diluted with a physiologically acceptable solvent, for example, to a melphalan flufenamide concentration (excluding the mass of any counterion) of about 0.001 mg/mL to about 3 mg/mL (for example, 0.01 mg/mL to 2 mg/mL, 0.05 mg/mL to 1.4 mg/mL, 0.05 mg/mL to 1.2 mg/mL, 0.05 mg/mL to 1 mg/mL, 0.05 mg/mL to 0.9 mg/mL, 0.05 mg/mL to 0.8 mg/mL, 0.1 mg/mL to 0.4 mg/mL, or 0.1 mg/mL to 0.3 mg/mL), before administration to a subject.

Thus, in certain embodiments, the pharmaceutical preparation of the invention comprises melphalan flufenamide at a concentration of about 0.001 mg/mL to about 3 mg/mL (for example, 0.01 mg/mL to 2 mg/mL, 0.05 mg/mL to 1.4 mg/mL, 0.05 mg/mL to 1.2 mg/mL, 0.05 mg/mL to 1 mg/mL, 0.05 mg/mL to 0.9 mg/mL, 0.05 mg/mL to 0.8 mg/mL 0.1 mg/mL to 0.4 mg/mL, 0.1 mg/mL to 0.3 mg/mL, or 0.1 mg/mL to 0.16 mg/mL) (excluding the mass of any counterion).

In certain preferred embodiments, a pharmaceutical preparation of the invention may be diluted with a physiologically acceptable solvent, for example, to a melphalan flufenamide concentration (excluding the mass of any counterion) of about 0.05 mg/mL to 0.9 mg/mL, preferably or 0.1 mg/mL to 0.3 mg/mL, and more preferably about 0.1 mg/mL to 0.16 mg/mL, before administration.

Melphalan flufenamide (for example melphalan flufenamide in its hydrochloride salt form (Ex 1) or deuterated melflufen flufenamide, such as Ex 2) may be administered to a subject at a concentration of about 0.1 mg/mL to about 3 mg/mL (excluding the mass of any counterion). A commonly used dosage regimen in the clinic for Ex 1 requires the melphalan flufenamide concentration in the infusion solution to be between 0.1 mg/mL and 0.16 mg/mL. Thus, in certain embodiments, the pharmaceutical preparation of the invention comprises melphalan flufenamide at a concentration of about 0.1 mg/mL to about 0.16 mg/mL (excluding the mass of any counterion) (for example, 0.10 mg/mL to 0.16 mg/mL).

Compositions suitable for parenteral administration of the present invention are preferably suitable for administration by infusion or injection, and especially suitable for administration by intravenous infusion.

In certain embodiments, injection and infusion solutions of melphalan flufenamide may be prepared by diluting a lyophilised powder or a liquid pharmaceutical formulation of melphalan flufenamide that does not contain γCD or a pharmaceutically acceptable derivative of γCD (such as HPγCD) with one or more physiologically acceptable solvents or diluents that contain γCD or a pharmaceutically acceptable derivative of γCD (such as HPγCD). For example, the melphalan flufenamide may be a lyophilised powder comprising (or consisting essentially of) melphalan flufenamide and sucrose. For example, the lyophilised powder may comprise (or consist essentially of) melphalan flufenamide and sucrose at a weight ratio of melphalan flufenamide to sucrose of 1:50 (for example, one vial of lyophilised powder may contain 20 mg of melphalan flufenamide (i.e. 21.48 mg of melphalan flufenamide hydrochloride) and 1,000 mg sucrose). Or, for example, the melphalan flufenamide may be a liquid pharmaceutical formulation comprising (or consisting essentially of) melphalan flufenamide and propylene glycol and/or PEG.

For example, the liquid pharmaceutical formulation contain melphalan flufenamide at a concentration of 15 mg/mL to 25 mg/mL, and propylene glycol and/or PEG as a solvent.

In embodiments wherein the one or more physiologically acceptable solvents or diluents contain γCD or a pharmaceutically acceptable derivative of γCD (such as HPγCD), the γCD or the pharmaceutically acceptable derivative of γCD (such as HPγCD) may be present at a concentration of about 1 mg/mL to 10 mg/mL. For example, the γCD or the pharmaceutically acceptable derivative of γCD (such as HPγCD) is present at a concentration of about 1 mg/mL to 3 mg/mL, for example about 1 mg/mL, 1.5 mg/mL, 2 mg/mL, 2.5 mg/mL, 3 mg/mL, 4 mg/mL, or 5 mg/mL.

Alternatively or additionally, γCD or a pharmaceutically acceptable derivative of γCD (such as HPγCD) may be added directly to the injection or infusion solution following the reconstitution and/or dilution of melphalan flufenamide with a suitable physiologically acceptable solvent or diluent; or melphalan flufenamide may be added directly to the injection or infusion solution following the reconstitution and/or dilution of γCD or a pharmaceutically acceptable derivative of γCD (such as HPγCD) with a suitable physiologically acceptable solvent or diluent. Thus, the present invention also provides an injection or infusion solution comprising melphalan flufenamide, γCD or a pharmaceutically acceptable derivative of γCD (such as HPγCD), one or more physiologically acceptable solvent or diluent, and optionally one or more further excipient.

In embodiments wherein γCD or a pharmaceutically acceptable derivative of γCD (such as HPγCD) is added directly to the injection or infusion solution following the reconstitution and/or dilution of melphalan flufenamide with a suitable physiologically acceptable solvent or diluent; or melphalan flufenamide is added directly to the injection or infusion solution following the reconstitution and/or dilution of γCD or a pharmaceutically acceptable derivative of γCD (such as HPγCD) with a suitable physiologically acceptable solvent or diluent, the melphalan flufenamide may be in the form of a pharmaceutical preparation, for example a lyophilised powder or a liquid pharmaceutical formulation comprising melphalan flufenamide. In exemplary embodiments, the melphalan flufenamide may be in the form of a lyophilised powder pharmaceutical preparation comprising melphalan flufenamide and sucrose. For example, the sucrose may be present at a weight ratio (w/w) of sucrose to melphalan flufenamide of about 2:1 to about 100:1, about 1:2 to about 1:75, or about 1:4 to about 1:75. Preferably, the sucrose is present at a weight ratio (w/w) of sucrose to melphalan flufenamide of about 20:1 to about 75:1 (for example, about 30:1 to about 70:1, about 40:1 to about 60:1, about 45:1 to about 55:1, or about example 50:1). In another exemplary embodiment, the melphalan flufenamide may be in the form of a liquid pharmaceutical formulation pharmaceutical preparation comprising melphalan flufenamide, or a salt thereof, and a pharmaceutically acceptable organic solvent (typically, the pharmaceutically acceptable organic solvent is selected from the group consisting of propylene glycol, polyethylene glycol (PEG), and mixtures thereof; in certain embodiments the pharmaceutical preparation consists essentially of melphalan flufenamide, or a salt thereof, and propylene glycol).

In embodiments that provide an injection or infusion solution comprising melphalan flufenamide, γCD or a pharmaceutically acceptable derivative of γCD (such as HPγCD), one or more physiologically acceptable solvent or diluent, and optionally one or more further excipient, the injection or infusion solution preferably comprises γCD or a pharmaceutically acceptable derivative of γCD (such as HPγCD) at a weight ratio (w/w) of γCD or a pharmaceutically acceptable derivative of γCD (such as HPγCD) to melphalan flufenamide of about 1:1 to about 100:1, for example γCD or a pharmaceutically acceptable derivative of γCD (such as HPγCD) at a weight ratio (w/w) of γCD or a pharmaceutically acceptable derivative of γCD (such as HPγCD) to melphalan flufenamide of about 1:1 to about 50:1, or about 3:1 to about 50:1. In one embodiment, the injection or infusion solution comprises γCD or a pharmaceutically acceptable derivative of γCD (such as HPγCD) at a weight ratio (w/w) of γCD or a pharmaceutically acceptable derivative of γCD (such as HPγCD) to melphalan flufenamide of about 4:1 to about 40:1. In exemplary embodiments, the injection or infusion solution comprises γCD or a pharmaceutically acceptable derivative of γCD (such as HPγCD) at a weight ratio (w/w) of γCD or a pharmaceutically acceptable derivative of γCD (such as HPγCD) to melphalan flufenamide of about 4:1 to about 30:1, preferably about 4:1 to about 25:1. Thus, in certain preferred embodiments, the injection or infusion solution comprises γCD or a pharmaceutically acceptable derivative of γCD (such as HPγCD) at a weight ratio (w/w) of γCD or a pharmaceutically acceptable derivative of γCD (such as HPγCD) to melphalan flufenamide of about 10:1 to about 25:1. For example, the injection or infusion solution comprises γCD or a pharmaceutically acceptable derivative of γCD (such as HPγCD) at a weight ratio (w/w) of γCD or a pharmaceutically acceptable derivative of γCD (such as HPγCD) to melphalan flufenamide of about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, or about 25:1.

More preferably, the weight ratio (w/w) of γCD or a pharmaceutically acceptable derivative of γCD (such as HPγCD) to melphalan flufenamide is about 13:1 to about 25:1. In one preferred embodiment, the weight ratio (w/w) of γCD or a pharmaceutically acceptable derivative of γCD (such as HPγCD) to melphalan flufenamide is about 15:1 to about 23:1. For example, the weight ratio (w/w) of γCD or a pharmaceutically acceptable derivative of γCD (such as HPγCD) to melphalan flufenamide is about 15:1, about 18:1, or about 23:1. In another preferred embodiment, the weight ratio (w/w) of γCD or a pharmaceutically acceptable derivative of γCD (such as HPγCD) to melphalan flufenamide is about 20:1 to about 25:1. For example, the weight ratio (w/w) of γCD or a pharmaceutically acceptable derivative of γCD (such as HPγCD) to melphalan flufenamide is about 20:1, about 22:1, about 24:1, or about 25:1.

In certain other preferred embodiments, the weight ratio (w/w) of γCD or a pharmaceutically acceptable derivative of γCD (such as HPγCD) to melphalan flufenamide is about 15:1 to about 25:1. For example, the injection or infusion solution comprises γCD or a pharmaceutically acceptable derivative of γCD (such as HPγCD) at a weight ratio (w/w) of γCD or a pharmaceutically acceptable derivative of γCD (such as HPγCD) to melphalan flufenamide of about 15:1, about 16:1, about 17:1, about 18:1, about 19:1, about 20:1, about 21:1, about 22:1, about 23:1, about 24:1, or about 25:1. In one preferred embodiment, the weight ratio (w/w) of γCD or a pharmaceutically acceptable derivative of γCD (such as HPγCD) to melphalan flufenamide is about 16:1 to about 25:1. In a further preferred embodiment, the weight ratio (w/w) of γCD or a pharmaceutically acceptable derivative of γCD (such as HPγCD) to melphalan flufenamide is about 20:1 to about 25:1. For example, the weight ratio (w/w) of γCD or a pharmaceutically acceptable derivative of γCD (such as HPγCD) to melphalan flufenamide is about 20:1, about 22:1, about 24:1, or about 25:1.

### Additional Therapeutic Agents

Whilst melphalan flufenamide, or a salt thereof, may be present in the pharmaceutical preparation of the invention as the sole active ingredient, it is also possible for the pharmaceutical preparation of the invention to additionally contain one or more additional therapeutic agent(s). Such agents are known in the art. Examples of further therapeutic agents for use in the present invention include steroids (prednisone, prednisolone and dexamethasone), IMiDs (thalidomide, lenalidomide and pomalidomide), PIs (bortezomib, carfilzomib and ixazomib), histone deacetylase (HDAC) inhibitors (panobinostat), conventional chemotherapy (alkylators (e.g. melphalan, cyclophosphamide, bendamustine), doxorubicin), and monoclonal antibodies (for example anti-CD38 antibodies (daratumumab) and anti-SLAMF7 antibodies (elotuzumab)).

Thus, in certain embodiments, the pharmaceutical preparation of the invention comprises melphalan flufenamide, or a salt thereof, γCD, and one or more additional therapeutic agent(s).

For example, the pharmaceutical preparation of the invention may consist essentially of melphalan flufenamide, or a salt thereof, γCD or a pharmaceutically acceptable derivative of γCD (such as HPγCD), and one or more additional therapeutic agent(s).

Or, for example, the pharmaceutical preparation of the invention may consist essentially of melphalan flufenamide, or a salt thereof, γCD or a pharmaceutically acceptable derivative of γCD (such as HPγCD), one or more further excipient (for example, sucrose), and one or more additional therapeutic agent(s). Or, for example, the pharmaceutical preparation of the invention may consist essentially of melphalan flufenamide, or a salt thereof, γCD or a pharmaceutically acceptable derivative of γCD (such as HPγCD), propylene glycol and/or PEG, and one or more additional therapeutic agent(s).

The precise quantity and concentration of the additional therapeutic agent that may be present in a pharmaceutical preparation or formulation or composition of the invention may vary with the dosing schedule, the potency of the particular agent chosen, the age, size, sex and condition of the subject (typically a mammal, for example a human), the nature and severity of the disease or condition, and other relevant medical and physical factors. The skilled person can readily determine the quantity and concentration of the melflufen, or salt thereof, and optional one or more additional therapeutic agent(s) suitable for use according to the present invention.

### Treatments

The pharmaceutical preparations, formulations and compositions of the present invention find use as medicaments.

In certain embodiments, the pharmaceutical preparations, formulations and compositions of the invention find use in the treatment and/or prophylaxis of diseases or conditions that may be treated with conventional chemotherapy agents, for example, with an alkylator (e.g. melphalan, cyclophosphamide and bendamustine).

In certain embodiments, the pharmaceutical preparations, formulations and compositions of the invention find use in the treatment and/or prophylaxis of cancer, reducing tumour growth and/or killing tumour cells. Thus, the pharmaceutical preparations, formulations and compositions of the invention may be used for curing and/or prolonging the survival of patients afflicted with cancer diseases. The pharmaceutical preparations, formulations and compositions of the invention find particular use in the treatment or prophylaxis of cancer selected from the group consisting of multiple myeloma, breast cancer, lung cancer, ovarian cancer, glioblastoma, leukaemias and lymphomas.

In other embodiments, the pharmaceutical preparations, formulations and compositions of the invention find use in the treatment and/or prophylaxis of amyloidosis.

The amount of melphalan flufenamide which is required to achieve a therapeutic effect will vary with particular route of administration and the characteristics of the subject under treatment, for example the species, age, weight, sex, medical conditions, the particular disease and its severity, and other relevant medical and physical factors. An ordinarily skilled physician can readily determine and administer the effective amount of melphalan flufenamide required for treatment or prophylaxis of cancer.

The pharmaceutical preparations, formulations and compositions of the invention find utility in a method for treating a subject which comprises administering a pharmaceutically effective amount of a pharmaceutical preparation, formulations or composition of the invention to a subject. Typically, a pharmaceutical preparation of the invention is administered following dilution into physiologically acceptable solvent or diluent, such as a saline solution or glucose solution. In embodiments wherein the pharmaceutical preparation is a liquid formulation, the pharmaceutical preparation may be administered directly to the subject, for example without prior dilution, for example, without prior dilution with a physiologically acceptable solvent or diluent.

### Unit Doses

The pharmaceutical preparation of the invention may be provided as unit doses. Preferred unit doses are those containing a requisite dose of melphalan flufenamide, or salt thereof, suitable for use according to the present invention.

In preferred embodiments, the pharmaceutical preparation of the invention may be provided in a vial containing a unit dose of melphalan flufenamide, or salt thereof.

For example, the pharmaceutical preparation of the invention may be provided in a vial containing a unit dose of 10 to 800 mg of melphalan flufenamide (excluding the mass of any counterion), for example, the vial may contain a unit dose of 10 mg to 50 mg, 50 mg to 150 mg, 150 mg to 250 mg, 250 mg to 350 mg, 350 mg to 450 mg, 450 mg to 550 mg, 550 mg to 650 mg, or 650 mg to 750 mg of melflufen (excluding the mass of any counterion). Preferably, the vial contains a unit dose of 5 mg to 15 mg, 15 mg to 25 mg, 25 mg to 50 mg, 50 mg to 75 mg, 75 mg to 125 mg, 125 mg to 175 mg, or 175 mg to 225 mg of melphalan flufenamide, for example 20 mg, 30 mg, 40 mg, 50 mg, 100 mg, 150 mg and 200 mg of melphalan flufenamide (excluding the mass of any counterion). In certain embodiments, the vial contains a unit dose of 100 mg of melphalan flufenamide (excluding the mass of any counterion). In certain embodiments, the vial contains a unit dose of 20 mg of melphalan flufenamide (excluding the mass of any counterion). In certain other embodiments, the vial contains a unit dose of 40 mg of melphalan flufenamide (excluding the mass of any counterion).

### Kits

The present invention provides a kit suitable for the preparation of a pharmaceutical preparation according to the invention. A kit of the invention comprises melphalan flufenamide, or a salt(s) thereof, and γCD or a pharmaceutically acceptable derivative of γ-cyclodextrin (such as HPγCD). The melphalan flufenamide and γCD or a pharmaceutically acceptable derivative of γCD (such as HPγCD) in the kit may be provided together as a mixture or they may be provided separately, for example in separate vials that are later combined prior to use of the kit (for example combined to prepare a pharmaceutical preparation, formulation or composition of the invention prior to use in a method or use of the invention defined herein). For example, the melphalan flufenamide and γCD or a pharmaceutically acceptable derivative of γCD (such as HPγCD) in the kit may be provided separately in separate vials that are later combined prior to use of the kit (for example combined to prepare a pharmaceutical preparation, formulations or composition of the invention prior to use in a method or use of the invention defined herein).In one preferred embodiment, the present invention provides a kit comprising a first vial containing contains melphalan flufenamide and sucrose as a lyophilized powder (for example, 20 mg melphalan flufenamide (21.48 mg of melphalan flufenamide hydrochloride, i.e. Ex 1) and 1000 mg sucrose as a lyophilized powder), and a second vial containing γCD or a pharmaceutically acceptable derivative of γCD (such as HPγCD). The vials are provided separately to be later combined prior to use of the kit (for example combined to prepare a pharmaceutical preparation, formulations or composition of the invention prior to use in a method or use of the invention defined herein).

In certain embodiments, the kit of the invention additionally comprises one or more physiologically acceptable aqueous solvent or diluent, such as a glucose solution or saline solution, as described herein. Typically the physiologically acceptable aqueous solvent or diluent is provided separately to the melphalan flufenamide and γCD or a pharmaceutically acceptable derivative of γCD (such as HPγCD) in the kit; or the physiologically acceptable aqueous solvent or diluent is provided together as a mixture with the γCD or the pharmaceutically acceptable derivative of γCD (such as HPγCD) in the kit and the melphalan flufenamide is provided separately, for example the melphalan flufenamide provided in a separate vial that is later combined with a composition comprising γCD or a pharmaceutically acceptable derivative of γCD (such as HPγCD) and physiologically acceptable aqueous solvent or diluent prior to use of the kit (for example combined to prepare a composition of the invention prior to use in a method or use of the invention defined herein).

In certain embodiments, the kit of the invention additionally comprises one or more pharmaceutically acceptable organic solvents selected from the group consisting of propylene glycol and PEG. The one or more PEG compounds preferably being one or more of the PEG compounds described herein. The one or more pharmaceutically acceptable organic solvents in the kit may be provided together as a mixture with one or both of melphalan flufenamide and γCD or a pharmaceutically acceptable derivative of γCD (such as HPγCD), or may be provided separately and combined with the melphalan flufenamide and γCD or a pharmaceutically acceptable derivative of γCD (such as HPγCD), for example in separate vials that are later combined prior to use of the kit. For example, the one or more pharmaceutically acceptable organic solvents in the kit may be provided together as a mixture with γCD or a pharmaceutically acceptable derivative of γCD (such as HPγCD), and the melphalan flufenamide may be provided separately and combined with the one or more pharmaceutically acceptable organic solvents and γCD or a pharmaceutically acceptable derivative of γCD (such as HPγCD) mixture prior to use of the kit.

Alternatively, for example, the one or more pharmaceutically acceptable organic solvents in the kit may be provided together as a mixture with both of melphalan flufenamide and γCD or a pharmaceutically acceptable derivative of γCD (such as HPγCD).

In one embodiment of the invention, the kit of the invention additionally comprises one or more further therapeutic agents as described herein. The one or more one or more further therapeutic agents in the kit may be provided together as a mixture with one or both of melphalan flufenamide and γCD or a pharmaceutically acceptable derivative of γCD (such as HPγCD), or may be provided separately.

For the avoidance of doubt, the melphalan flufenamide, or salt thereof, γCD or a pharmaceutically acceptable derivative of γCD (such as HPγCD), optional physiologically acceptable aqueous solvent or diluent, optional one or more pharmaceutically acceptable organic solvent, and optional one or more further therapeutic agent, are present in a kit according to the present invention in a form and quantity suitable for the preparation of a pharmaceutical preparation according to the invention. The skilled person can readily determine a quantity of the melflufen, or a salt thereof, pharmaceutically acceptable organic solvent, and optional one or more further therapeutic agents, suitable for the use according the present invention.

The present invention also provides kits suitable for the preparation of injection and infusion solutions comprising melphalan flufenamide and γCD or a pharmaceutically acceptable derivative of γCD (such as HPγCD). Such a kit comprises melphalan flufenamide, or a salt(s) thereof, γCD or a pharmaceutically acceptable derivative of γCD (such as HPγCD), and one or more physiologically acceptable aqueous solvent or diluent, such as a glucose solution or saline solution, as described herein. The melphalan flufenamide, γCD or a pharmaceutically acceptable derivative of γCD (such as HPγCD), and one or more physiologically acceptable aqueous solvent or diluent may be provided as a mixture or separately. For example, the melphalan flufenamide and γCD or a pharmaceutically acceptable derivative of γCD (such as HPγCD) may be provided together as a mixture and the one or more physiologically acceptable aqueous solvent or diluent may be provided separately. Alternatively, the γCD or the pharmaceutically acceptable derivative of γCD (such as HPγCD) and one or more one or more physiologically acceptable aqueous solvent or diluent may be provided as a mixture (i.e. provided as a composition comprising γCD or a pharmaceutically acceptable derivative of γCD (such as HPγCD) and one or more one or more physiologically acceptable aqueous solvent or diluent) and the melphalan flufenamide may be provided separately.

Preferably the pharmaceutically acceptable aqueous solvent is 5% glucose solution (w/v in water) or a 0.9% sodium chloride solution (w/v in water). Preferably, such solutions are suitable for intravenous injection and/or infusion to a subject.

In such kits, preferably the melphalan flufenamide, or a salt(s) thereof, and γCD or a pharmaceutically acceptable derivative of γCD (such as HPγCD) are provided as a pharmaceutical preparation of the invention, for example a lyophilised powder of the invention or a liquid pharmaceutical formulation of the invention. As such, the present invention provides a kit suitable for the preparation of injection and infusion solutions comprising a pharmaceutical preparation of the invention (for example a lyophilised powder of the invention or a liquid pharmaceutical formulation of the invention), and one or more physiologically acceptable aqueous solvent or diluent, such as a glucose solution or saline solution, as described herein.

Alternatively, the present invention provides a kit suitable for the preparation of injection and infusion solutions comprising a pharmaceutical preparation comprising melphalan flufenamide, or a salt(s) thereof (for example a lyophilized pharmaceutical preparation comprising melphalan flufenamide, or a salt(s) thereof, and sucrose), a composition comprising γCD or a pharmaceutically acceptable derivative of γCD (such as HPγCD), and one or more physiologically acceptable aqueous solvent or diluent, such as a glucose solution or saline solution, as described herein.

For example, the present invention provides a kit suitable for the preparation of injection and infusion solutions comprising a lyophilized pharmaceutical preparation comprising melphalan flufenamide, or a salt(s) thereof, and sucrose (for example, 20 mg melphalan flufenamide (21.48 mg of melphalan flufenamide hydrochloride, i.e. Ex 1) and 1000 mg sucrose as a lyophilized powder), a composition comprising γCD or a pharmaceutically acceptable derivative of γCD (such as HPγCD), and one or more physiologically acceptable aqueous solvent or diluent, such as a glucose solution or saline solution, as described herein.

In one embodiment of the invention, the kit of the invention suitable for the preparation of injection and infusion solutions additionally comprises one or more further therapeutic agents as described herein.

Preferably, the kit of the invention comprises instructions, for example instructions that instruct a user to admix a stated amount of melphalan flufenamide with a stated amount of γCD or a pharmaceutically acceptable derivative of γCD (such as HPγCD).

Additionally, or alternatively, the instructions may instruct a user to admix a stated amount of a melphalan flufenamide and γCD or a pharmaceutically acceptable derivative of γCD (such as HPγCD) mixture, with a stated amount of one or more pharmaceutically acceptable organic solvent and/or stated amount of one or more physiologically acceptable aqueous solvent.

Additionally, or alternatively, the instructions may instruct a user to admix a stated amount melphalan flufenamide with a stated amount of one or more pharmaceutically acceptable organic solvent comprising γCD or a pharmaceutically acceptable derivative of γCD (such as HPγCD) and/or stated amount of one or more physiologically acceptable aqueous solvent comprising γCD or a pharmaceutically acceptable derivative of γCD (such as HPγCD).

Additionally, or alternatively, the instructions may instruct a user to admix a stated amount melphalan flufenamide with a stated amount of one or more pharmaceutically acceptable organic solvent and/or stated amount of one or more physiologically acceptable aqueous solvent, and then to admix a stated amount of γCD or a pharmaceutically acceptable derivative of γCD (such as HPγCD) to the mixture of melphalan flufenamide, one or more pharmaceutically acceptable organic solvent and/or one or more physiologically acceptable aqueous solvent.

Such instructions may also provide guidance on the storage conditions and/or administration instructions.

For the avoidance of doubt, the melphalan flufenamide, or salt thereof, γCD or a pharmaceutically acceptable derivative of γCD (such as HPγCD), one or more physiologically acceptable aqueous solvent or diluent, and optional one or more further therapeutic agent, are present in a kit according to the present invention in a form and quantity suitable for the preparation of a solution suitable for use as an injection or infusion solution. The skilled person can readily determine a quantity of the melflufen, or a salt thereof, γCD or a pharmaceutically acceptable derivative of γCD (such as HPγCD), physiologically acceptable aqueous solvent, and optional one or more further therapeutic agents, suitable for the use according the present invention.

Finally, in embodiments of the invention wherein a kit is provided, a kit of the present invention may comprises γCD or a pharmaceutically acceptable derivative of γCD (such as HPγCD) at a weight ratio (w/w) of γCD or a pharmaceutically acceptable derivative of γCD (such as HPγCD) to melphalan flufenamide of about 1:1 to about 100:1, for example γCD or a pharmaceutically acceptable derivative of γCD (such as HPγCD) at a weight ratio (w/w) of γCD or a pharmaceutically acceptable derivative of γCD (such as HPγCD) to melphalan flufenamide of about 4:1 to about 80:1, about 4:1 to about 50:1, or about 4:1 to about 30:1, wherein the amount of melphalan flufenamide is the amount present in the kit. In exemplary embodiments, the pharmaceutical preparations of the present invention comprise γCD or a pharmaceutically acceptable derivative of γCD (such as HPγCD) at a weight ratio (w/w) of γCD or a pharmaceutically acceptable derivative of γCD (such as HPγCD) to melphalan flufenamide of about 4:1 to about 25:1, preferably about 5:1 to about 25:1, wherein the amount of melphalan flufenamide is the amount present in the kit.

In certain preferred embodiments, the kits of the present invention comprise γCD or a pharmaceutically acceptable derivative of γCD (such as HPγCD) at a weight ratio (w/w) of γCD or a pharmaceutically acceptable derivative of γCD (such as HPγCD) to melphalan flufenamide of about 10:1 to about 25:1, wherein the amount of melphalan flufenamide is the amount present in the kit. For example, the kits of the present invention may comprise γCD or a pharmaceutically acceptable derivative of γCD (such as HPγCD) at a weight ratio (w/w) of γCD or a pharmaceutically acceptable derivative of γCD (such as HPγCD) to melphalan flufenamide of about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, or about 25:1. In one preferred embodiment, the weight ratio (w/w) of γCD or a pharmaceutically acceptable derivative of γCD (such as HPγCD) to melphalan flufenamide is about 10:1 to about 23:1, wherein the amount of melphalan flufenamide is the amount present in the kit. For example, the weight ratio (w/w) of γCD or a pharmaceutically acceptable derivative of γCD (such as HPγCD) to melphalan flufenamide is about 15:1, about 18:1, or about 23:1. In another preferred embodiment, the weight ratio (w/w) of γCD or a pharmaceutically acceptable derivative of γCD (such as HPγCD) to melphalan flufenamide is about 15:1 to about 25:1 (for example 20:1 to 25:1), wherein the amount of melphalan flufenamide is the amount present in the kit. For example, the weight ratio (w/w) of γCD or a pharmaceutically acceptable derivative of γCD (such as HPγCD) to melphalan flufenamide is about 15:1, about 20:1, about 22:1, about 24:1 or about 25:1.

### EQUIVALENTS

The invention has been described broadly and generically herein. Those of ordinary skill in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. The applicant reserves the right physically to incorporate into this application any and all materials and information from any such articles, patents, patent applications, or other physical and electronic documents.

The following Examples illustrate the invention.

### Examples

### Example 1: The stabilising effect of γ-cyclodextrin on melphalan flufenamide

The ability of γ-cyclodextrin (γCD) to stabilise melphalan flufenamide in aqueous solution was investigated and compared with other excipients.

In this example, melphalan flufenamide was used in its hydrochloride salt form (referred to hereinafter as Example compound 1-Ex 1). Methods for the preparation of Ex 1 are disclosed in WO 01/96367 and WO 2016/180740, which are incorporated herein by reference.

### Sample preparation

The following excipients were used: trehalose dihydrate, sucrose, polysorbate 80, α-cyclodextrin, β-cyclodextrin, propylene glycol, 2-hydroxypropyl-β-cyclodextrin, and γCD. Samples were prepared at a weight ratio (w/w) of excipient to Ex 1 of 4:1 (w/w).

A stock solution of each excipient was prepared in water (10 mg/mL). An aliquot of 400 μL (4 mg) of each stock solution was then transferred to a separate transparent 4 mL vial. The vials containing the stock solutions were then cooled and lyophilized using a Christ Alpha 2-4 LSC Plus instrument.

1 mL of a 1 mg/mL solution of Ex 1 in water/t-butanol (1:1 v/v) was added to each of the vials containing the lyophilised excipients to give a mixture of excipient to Ex 1 in a weight ratio (w/w) of 4:1. The mixtures were cooled and lyophilized overnight.

1 mL of a 5% glucose solution (w/v in water) containing 0.08 mg/mL 3-methoxybenzoic as an internal standard was added to each vial containing a lyophilized Ex 1-excipient mixture. The vials were then mixed by shaking for 15 seconds, and then shaken occasionally for 5 minutes. The solutions were transferred to Whatman® Mini-UniPrep® syringeless filters and filtered according to the manufacturer's instructions. The filtrate was transferred to a glass vial to prevent further dissolution. These filtrates represent the starting concentrations of melphalan flufenamide for each solution (i.e. the to samples). The to samples were analysed by HPLC as described below and then stored at 25° C. to 30° C. for 22 hours.

Sample analysis:

The amount of Ex 1 in solution in each sample was determined by HPLC analysis using a calibration curve for Ex 1. The percentage of Ex 1 remaining in solution following 18 to 20 hours of storage was calculated relative to the amount of Ex 1 present in the corresponding to sample for each excipient tested.

HPLC analysis of each sample was performed using an Agilent 1100 series Liquid Chromatograph/Mass Selective Detector (MSD) (Single Quadrupole) equipped with an electrospray interface and a UV diode array detector. Analyses were performed using an ACE 3 C8 (3.0×50 mm) column with a gradient of 5 to 95% acetonitrile, 0.1% aqueous TFA over 3 minutes with a flow rate of 1 mL/min. An injection volume of 2 μL was used.

### Results

Table 1 shows the percentage of Ex 1 remaining in each solution after storage at 25° C. to 30° C. for 22 hours.

TABLE 1

| | % of Ex 1 remaining in solution after |
|---|---|
| Excipient in solution | storage at 30° C. for 22 hours |
| γCD | 69 |
| polysorbate 80 | 39 |
| β-cyclodextrin hydrate | 23 |
| 2-hydroxypropyl-β-cyclodextrin | 17 |
| α-cyclodextrin | 17 |
| propylene glycol | 11 |
| sucrose | 10 |
| trehalose dihydrate | 10 |

In the absence of an excipient, Ex 1 has a degradation rate of around 8 to 10% per hour at room temperature (RT). As shown above, Ex 1 was found to display good stability in a γCD 5% glucose solution (w/v in water), with 69% of Ex 1 remaining in solution after storage at 25° C. to 30° C. for 22 hours.

### Example 2: The effect of γ-cyclodextrin on the rate of dissolution of melphalan flufenamide

In a clinical setting, it is important that the dissolution of the melphalan flufenamide is not negatively affected by any excipients present in the drug product. The rate of dissolution of melphalan flufenamide in the presence of the excipients tested in Example 1 was therefore investigated. In this example, melphalan flufenamide was used in its hydrochloride salt form (i.e. Ex 1).

Sample preparation:

Samples were prepared at a ratio of Ex 1 (free base) to excipient of 1:4 (w/w). A stock solution of each excipient was prepared in water (10 mg/mL). An aliquot of 400 μL (4 mg) of each stock solution was then transferred to a separate transparent 4 mL vial. The vials containing the stock solutions were then cooled and lyophilized using a Christ Alpha 2-4 LSC Plus instrument.

1 mL of a solution of 1 mg/mL solution of Ex 1 in water/t-butanol (1:1 v/v) was added to each of the vials containing the lyophilised excipients to give a mixture of Ex 1 to excipient in a ratio of 1:4 (w/w). The mixtures were cooled and lyophilized overnight.

A 5% glucose solution (w/v in water) containing 0.08 mg/mL 3-methoxybenzoic as an internal standard was added to each vial containing a lyophilized Ex 1-excipient mixture. The vials were then mixed by shaking for 15 seconds. Approximately half of the solution was then transferred to a Whatman® Mini-UniPrep© syringeless filter and filtered according to the manufacturer's instructions. The filtrate was immediately transferred to a glass vial to prevent further dissolution.

Sample analysis:

The amount of Ex 1 in solution in each sample was determined by HPLC analysis using a calibration curve for Ex 1. HPLC analysis of each sample was performed using the same method as described in Example 1.

### Results:

The amounts of Ex 1 dissolved in each sample are shown in Table 2.

TABLE 2

| | Ex 1 concentration after |
|---|---|
| Excipient in solution | 15 second dissolution, mg/mL |
| γCD | 0.68 |
| α-cyclodextrin | 0.62 |
| polysorbate 80 | 0.51 |
| β-cyclodextrin | 0.43 |
| 2-hydroxypropyl-β-cyclodextrin | 0.37 |
| trehalose dihydrate | 0.26 |
| sucrose | 0.21 |
| propylene glycol | 0.11 |

As shown above, it was found that Ex 1 displayed the highest initial rate of dissolution in the solution containing γCD, with 68% of Ex 1 being dissolved within the first 15 seconds of mixing. In the absence of an excipient, Ex 1 has solubility of <0.4 mg/mL in aqueous solution. The data shown above therefore show that Ex 1 displays very favourable dissolution speed and solubility in the presence of γCD.

### Example 3: The stabilising effect of γ-cyclodextrin on melphalan flufenamide and Example compound 2 in a non-aqueous liquid formulation

The effect of γCD on the stability of melphalan flufenamide and Example compound 2 (Ex 2) in the form of liquid formulations using propylene glycol as solvent was investigated.

### Sample preparation

### Example compound 2 has the following structure

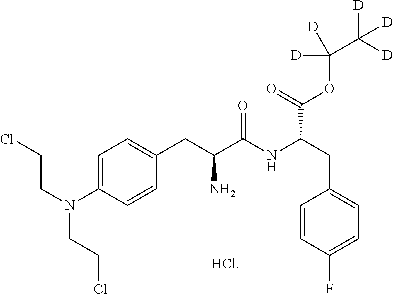 [SMILES: [2H]C([2H])([2H])C([2H])([2H])OC(=O)[C@H](Cc1ccc(F)cc1)NC(=O)[C@@H](N)Cc1ccc(N(CCCl)CCCl)cc1 ; "HCl."]

Methods for the preparation of Ex 2 are disclosed in WO 2020/079165, which is incorporated herein by reference. In this example, melphalan flufenamide was used in its hydrochloride salt form (i.e. Ex 1) and Ex 2 was used in its hydrochloride salt form to prepare the liquid preparations of those compounds.

Stock solutions containing 16 mg/mLγCD in 0. 9% sodium chloride solution (w/v in water) or 5% glucose solution (w/v in water); and liquid formulations of Ex 1 (20 mg/mL free base) or Ex 2 (20 mg/mL free base) in propylene glycol were prepared.

Using the stock solutions and liquid formulations, the following samples were prepared:
1.2 mg/mL Ex 1 (free base) or Ex 2 (free base), 6% propylene glycol, and 15-16 mg/mLγCD 5% glucose solution (w/v in water);
0.6 mg/mL Ex 1 (free base) or Ex 2 (free base), 3% propylene glycol, and 15-16 mg/mLγCD 5% glucose solution (w/v in water);
0.2 mg/ml Ex 1 (free base) or Ex 2 (free base), 1% propylene glycol, and 15-16 mg/mLγCD 5% glucose solution (w/v in water);
1.2 mg/mL Ex 1 (free base) or Ex 2 (free base), 6% propylene glycol, and 15-16 mg/mLγCD 0.9% sodium chloride solution (w/v in water);
0.6 mg/mL Ex 1 (free base) or Ex 2 (free base), 3% propylene glycol, and 15-16 mg/ml γCD 0.9% sodium chloride solution (w/v in water); and
0.2 mg/mL Ex 1 (free base) or Ex 2 (free base), 1% propylene glycol, and 15-16 mg/ml γCD 0.9% sodium chloride solution (w/v in water).

In brief, samples containing 1.2 mg/mL Ex 1 (free base) or Ex 2 (free base) were prepared by adding 60 μL of the Ex 1 or Ex 2 liquid formulation into 940 μL of a γCD stock solution; samples containing 0.6 mg/mL Ex 1 (free base) or Ex 2 (free base) were prepared by adding 30 μL of the Ex 1 or Ex 2 liquid formulation into 970 μL of a γCD stock solution; and samples containing 0.2 mg/mL Ex 1 (free base) or Ex 2 (free base) were prepared by adding 20 μL of the Ex 1 or Ex 2 liquid formulation into 1980 μL of a γCD stock solution.

The samples were mixed by vortex for 10 seconds and then filtered through a 0.45 μm PTFE filter. The filtrates were analysed by HPLC immediately after sample preparation (i.e. at t₀), and then after 2 hours, 5 hours, 7 hours or 18 hours of storage at RT (approximately 23° C.).

Sample analysis:

The Ex 1 (free base) or Ex 2 (free base) concentrations in the to samples and samples stored for 2 hours, 5 hours, 7 hours or 18 hours at RT were determined by HPLC analysis using the method described in Example 4.

### Results

Ex 2 was found to display poor stability in the control solutions (6% propylene glycol and saline only or 6% propylene glycol and 5% glucose only). γCD improved the stability of Ex 2 when used in a saline solution or 5% glucose solution at a weight ratio (w/w) of γCD to melphalan flufenamide of about 16:1, about 30:1, and about 80:1. FIG. 1 shows the stabilising effect of γCD on melphalan flufenamide when used at a weight ratio (w/w) of γCD to melphalan flufenamide of about 16:1. The stability enhancing properties of γCD were found to be independent of the propylene glycol concentration. Ex 1 and Ex 2 were found to display comparable stability and solubility, and have comparable HPLC retention times.

### Example 4: The stabilising effect of γ-cyclodextrin on a lyophilised melphalan flufenamide preparation made up in aqueous solution

In August 2022, the European Medicines Agency approved a melphalan flufenamide hydrochloride preparation for use to treat adults with multiple myeloma (a cancer of the bone marrow) when the cancer has not responded to previous treatments (refractory). It is used in combination with dexamethasone (an anti-inflammatory medicine) in adults who have received at least three prior therapies, including an immunomodulatory agent, a proteasome inhibitor and an antiCD38 antibody, and whose disease has worsened since the last treatment. For patients who have had an autologous stem cell transplantation (a procedure where the patient's bone marrow is cleared of cells and replaced by stem cells from the patients themselves), it can be used if the time from transplantation to when the cancer comes back is at least three years. The approved melphalan flufenamide preparation is sold in Europe under the name PEPAXTI© (another trade name for melphalan flufenamide hydrochloride is PEPAXTO®). A single-dose vial of the approved melphalan flufenamide preparation PEPAXTI® (hereinafter referred to as the melphalan flufenamide drug product) contains 20 mg melphalan flufenamide (21.48 mg of melphalan flufenamide hydrochloride, i.e. Ex 1) and 1000 mg sucrose as a lyophilized powder. PEPAXTO® has an identical formulation. A series of experiments were performed to identify the optimum concentration of γCD for improving the stability of the melphalan flufenamide drug product in an aqueous solution.

Sample preparation:

A stock solution of 23 mg/mL γCD 5% glucose solution (w/v in water) was used to prepare working solutions containing 1 mg, 5 mg, 10 mg, 15 mg, 18 mg, 20 mg or 23 mg/mL γCD.

Approximately 50 mg of the melphalan flufenamide drug product (containing approximately 1 mg of melphalan flufenamide) was transferred to 8 different 4 mL clear glass vials, followed by addition of 1 mL of a γCD working solution. The solutions were stirred for 5 minutes. The solutions were then filtered through a 0.45 μm PTFE filter. The filtrates were analysed by HPLC immediately after sample preparation (i.e. at t₀), and then after 2 hours, 5 hours, 7 hours or 18 hours of storage at RT (approximately 23° C.).

Sample analysis:

HPLC analysis was performed using an Agilent 1260 series HPLC. Analyses were performed using a Poroshell column 3×50 mm EC-C18 column with a gradient of 5% to 97% acetonitrile, 0.1% formic acid over 3 min and a flow of 1 mL/min. An injection volume 0.2 μL was used. The melphalan flufenamide concentration in each sample was determined by HPLC analysis using a calibration curve for melphalan flufenamide.

### Results:

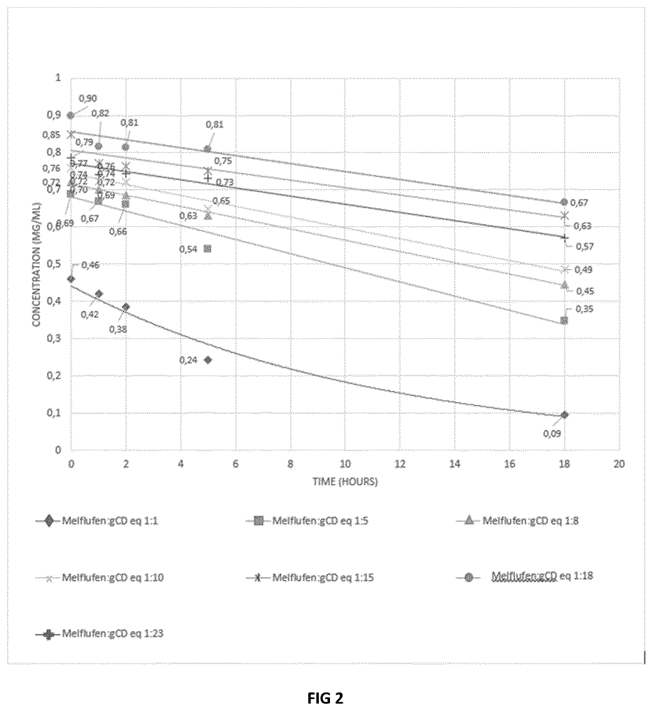

As shown in FIG. 2, melphalan flufenamide was stabilised by solutions containing 1 to 23 equivalents of γCD and 1 equivalent of melphalan flufenamide ratios (w/w). Melphalan flufenamide was most stable in the solutions containing 15, 18 and 23 equivalents of γCD and 1 equivalent of melphalan flufenamide (w/w).

### Example 5: The stabilising effect of γ-cyclodextrin on a melphalan flufenamide infusion solution

Administration of the melphalan flufenamide drug product to a patient in the clinic is generally performed by intravenous infusion over a period of 30 minutes using a central venous access device. The melphalan flufenamide concentration in the infusion solution is usually 0.1 mg/mL to 0.16 mg/mL. Because melphalan flufenamide degrades in aqueous solution, the steps for preparing the infusion solution must be performed quickly and infusion to a patient must begin within 60 minutes of the reconstitution step of the lyophilized powder. Storing the prepared infusion solution at 2° C. to 8° C. allows for delayed administration which must not exceed 6 hours of the reconstitution step. The stabilising effect of γCD on melphalan flufenamide contained in an infusion solution was investigated and compared to the stability of melphalan flufenamide in an infusion solution not containing γCD.

Samples preparation:

A control infusion solution containing no γCD was prepared in accordance with the following method, which is in line with the protocol set out in the U.S. Prescribing Information (USPI) for PEPAXTO® dated February 2021:

TABLE 3

| | |
|---|---|
| Step 1 | Place two melphalan flufenamide drug product vials at RT for |
| | at least 30 minutes; |
| Step 2 | Shake the vials vigorously or vortex to disintegrate the |
| | lyophilized melphalan flufenamide drug product powder cake |
| | into a loose powder; |
| Step 3* | Reconstitute each vial with 40 mL of 5% glucose solution |
| | (w/v in water) to obtain a final concentration of 0.5 mg/mL |
| | melphalan flufenamide. Ensure the 5% glucose solution is |
| | at room temperature before the reconstitution step (i.e. at |
| | 20° C. to 25° C.). Shake the vial(s) vigorously until the |
| | solution is clear. Let the vial(s) stand to allow air bubbles |
| | to dissipate to confirm a clear solution; |
| Step 4* | Withdraw 80 mL from a refrigerated (i.e. at 2° C. to 8° C.) |
| | 250 mL infusion bag of 0.9% sodium chloride solution |
| | (w/v in water). Discard the withdrawn 80 mL; and |
| Step 5* | Withdraw 40 mL of reconstituted melphalan flufenamide |
| | drug product solution from each vials (total 80 mL) and |
| | transfer into the intravenous bag from step 4 to obtain a final |
| | concentration of 0.16 mg/mL melphalan flufenamide. |
| | Gently invert the infusion bag to mix the solution. Do not |
| | shake. Check that the infusion solution is clear and colourless |
| | to pale yellow. |

*Steps 3 to 5 must be completed within 30 minutes.

An infusion solution containing γCD was prepared according to steps 1 to 5 above, except that step 2 included a step of adding 320 mg of γCD into each vial (i.e. 1:16 ratio w/w of melphalan flufenamide to γCD) followed by mixing the powders by vortex. Steps 3 to 5 were then carried out as set out above.

Immediately after preparation of each infusion solution, 4 mL was withdrawn from the infusion bag and the melphalan flufenamide content was determined by HPLC analysis. These samples represent each infusion solution at the start of the stability study (i.e. the to samples). Each infusion bag was then stored at 5° C. 4 mL samples of infusion solution were removed from each infusion bag after 1 hour, 3 hours, 5 hours, 23 hours, 27 hours, 29 hours and 43 hours of storage at 5° C. The melphalan flufenamide content of each sample was determined by HPLC analysis.

Sample analysis:

The melphalan flufenamide content of each sample of infusion solution was determined by HPLC analysis using the method described in Example 5.

### Results:

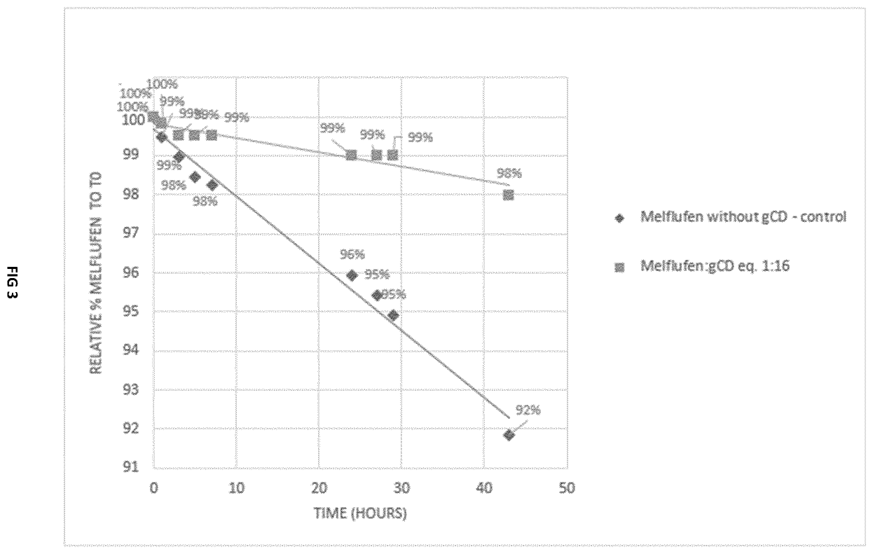

After 24 hours of storage at 5° C., the melphalan flufenamide content of the control infusion solution (i.e. the solution not containing γCD) was found to have decreased by approximately 7% after 24 hours of storage at 5° C. relative to the melphalan flufenamide content of the to samples. After 43 hours of storage at 5° C., the melphalan flufenamide content of the control infusion solution was found to have decrease by approximately 10% relative to relative to the melphalan flufenamide content of the to samples. In contrast, after 43 hours of storage at 5° C., the melphalan flufenamide content of the infusion solution containing γCD was found to have only decreased by approximately 2% to 3% relative to relative to the melphalan flufenamide content of the to samples—see FIG. 3.

### Example 6: The stabilising effect of a derivative of γ-cyclodextrin on a melphalan flufenamide infusion solution

Stock solutions of 16 mg/mL hydroxypropyl-γ-cyclodextrin in 5% glucose and in 0.9% saline were prepared. 20 mg/mL (free base) solution of the compound of Example 2 in propylene glycol was prepared. The hydroxypropyl-γ-cyclodextrin was obtained from Sigma-Aldrich (product No 779229) and it had a degree of substitution of 0.6 (corresponding to an average number of hydroxypropyl groups per γCD molecule of about 4.7).

To a 2 mL clear vial was added the amounts of each stock solution according to the Table 4 below.

TABLE 4

| | | Ex 2 stock | | | Percentage |
|---|---|---|---|---|---|
| | | solution | | | propylene |
| | Conc. | 20 mg/mL | | Medium | glycol |
| Entry | (mg/mL) | (μL) | Medium | (μL) | (%) |
| 1 | 1.2 | 60 | 5% Glucose | 940 | 6 |
| 2 | 0.6 | 30 | 5% Glucose | 970 | 3 |
| 3 | 0.2 | 10 | 5% Glucose | 990 | 1 |
| 4 | 1.2 | 60 | 0.9% saline | 940 | 6 |
| 5 | 0.6 | 30 | 0.9% saline | 970 | 3 |
| 6 | 0.2 | 10 | 0.9% saline | 990 | 1 |
| 7 | 1.2 | 60 | 16 mg/mL HPγCD | 940 | 6 |
| | | | in 5% Glucose | | |
| 8 | 0.6 | 30 | 16 mg/mL HPγCD | 970 | 3 |
| | | | in 5% Glucose | | |
| 9 | 0.2 | 10 | 16 mg/mL HPγCD | 990 | 1 |
| | | | in 5% Glucose | | |
| 10 | 1.2 | 60 | 16 mg/mL HPγCD | 940 | 6 |
| | | | in 0.9% saline | | |
| 11 | 0.6 | 30 | 16 mg/mL HPγCD | 970 | 3 |
| | | | in 0.9% saline | | |
| 12 | 0.2 | 10 | 16 mg/mL HPγCD | 990 | 1 |
| | | | in 0.9% saline | | |

The samples were stored at room temperature and analysed after 0, 2, 5 and 23 h on HPLC. The degradation of the compound was reported relative to the area under the curve at time=0 h. The percentage figure in Table 5 below represents the proportion of remaining compound of Ex 2.

TABLE 5

| Entry | 2 h | 5 h | 23 h |
|---|---|---|---|
| 1 | 85% | 65% | 17% |
| 2 | 82% | 60% | 12% |
| 3 | 80% | 56% | 9% |
| 4 | 98% | 89% | 56% |
| 5 | 31% | 21% | 15% |
| 6 | 78% | 60% | 39% |
| 7 | 90% | 85% | 61% |
| 8 | 89% | 80% | 60% |
| 9 | 88% | 75% | 49% |
| 10 | 99% | 97% | 88% |
| 11 | 98% | 95% | 87% |
| 12 | 99% | 96% | 89% |

In entry 4, 5 and 6 there was precipitation of the compound.

Analytical HPLC was carried out on an Agilent Series 1100 system using a Kinetex XB C18 (2.6 μm, 3.0×50 mm) column with 0.1% TFA in MiliQ H2O/MeCN as mobile phase (flow 1 mL/min). 5-97% MeCN in 3 minutes.

It is seen in the table that for each of the pairs (1-7, 2-8, 3-9, 4-10, 5-11 and 6-12), the solution including the hydroxypropyl-γ-cyclodextrin had more of the compound of Example 2 remaining at the end of the storage. That is to say that the solutions including the hydroxypropyl-γ-cyclodextrin had better stability.

### Example 7: The stabilising effect of a derivative of γ-cyclodextrin on a melphalan flufenamide infusion solution at different weight ratios

A stock solution of 10 wt % of hydroxypropyl-γ-cyclodextrin in saline was prepared. A stock solution of the compound of Example 2 in propylene glycol (20 mg/mL, free base) was prepared.

To a 4 mL clear vial were added the stock solution of Example 2 (20 μL), the 10 wt % stock solution of hydroxypropyl-γ-cyclodextrin in saline and saline to make different ratios of Example 2 and excipient according to Table 6 below. The concentration of Ex 2 in the final solutions was 0.2 mg/mL.

TABLE 6

| | 10 wt % | | | | |
|---|---|---|---|---|---|
| | HPγCD in | | | molecular | |
| Entry | saline (μl) | Saline (μL) | weight ratio | ratio | Comment |
| 1 | 16 | 1964 | 4 | 1.28 | not soluble |
| 2 | 32 | 1948 | 8 | 2.55 | not soluble |
| 3 | 48 | 1932 | 12 | 3.83 | not soluble |
| 4 | 64 | 1916 | 16 | 5.11 | not soluble |
| 5 | 80 | 1900 | 20 | 6.38 | |
| 6 | 96 | 1884 | 24 | 7.66 | |
| 7 | 112 | 1868 | 28 | 8.94 | |
| 8 | 128 | 1852 | 32 | 10.21 | |
| 9 | 144 | 1836 | 36 | 11.49 | |
| 10 | 160 | 1820 | 40 | 12.77 | |

The samples were analysed after 0 h and the samples were stored at room temperature or in the fridge and analysed after 18 h. The remaining API (i.e. the remaining melphalan flufenamide) was reported relative to the area under the curve at t=0 h.

Analytical HPLC was carried out on an Agilent Series 1100 system using a Kinetex XB C18 (2.6 μm, 3.0×50 mm) column with 0.1% TFA in MiliQ H2O/MeCN as mobile phase (flow 1 mL/min). 5-97% MeCN in 3 minutes.

The results are shown in Table 7 below. It is seen in Table 7 that good stability was achieved for each of the weight ratios. It is noteworthy that especially good stability was achieved for weight ratios of 1:32 to 1:40.

TABLE 7

| | | Amount API | |
|---|---|---|---|
| | | 18 h at room | |
| | | temperature | Amount API |
| Entry | weight ratio | (%) | 18 h in fridge (%) |
| 5 | 1:20 | 78.69 | 94.78 |
| 6 | 1:24 | 83.79 | 98.70 |
| 7 | 1:28 | 81.58 | 93.49 |
| 8 | 1:32 | 86.01 | 98.27 |
| 9 | 1:36 | 87.19 | 99.49 |
| 10 | 1:40 | 88.12 | 99.11 |

### Example 8: The stabilising effect of a derivative of γ-cyclodextrin on a melphalan flufenamide infusion solution at different weight ratios and concentrations

Stock solutions of 10 wt % of hydroxypropyl-γ-cyclodextrin in saline, and 10 wt % of hydroxypropyl-γ-cyclodextrin in 5% glucose, were prepared. A stock solution of Ex 2 in propylene glycol (20 mg/mL, free base) was prepared.

The stock solution of Ex 2 (100 μL), and the 10 wt % stock solution of hydroxypropyl-γ-cyclodextrin in saline or the 10 wt % stock solution of hydroxypropyl-γ-cyclodextrini in 5% glucose (see “Medium” in Table 8), were added to clear vials according to Table 8. Additional 0.9% saline or 5% glucose were added to obtain the concentrations indicated in Table 8.

TABLE 8

| | | | Stock | | | | |
|---|---|---|---|---|---|---|---|
| | Amount | | solution | | Conc | Amount | |
| | Ex 2 | | HPγCD | Medium | Ex 2 | HPγCD | Weight ratio |
| Entry | (mg) | Medium | (mL) | (mL) | (mg/mL) | (mg) | Ex 2:HPγCD |
| 1 | 2 | 5% glucose | 1.000 | 0 | 1.82 | 100 | 50 |
| 2 | 2 | 0.9% saline | 1.000 | 0 | 1.82 | 100 | 50 |
| 3 | 2 | 0.9% saline | 0.500 | 16.167 | 0.12 | 50 | 25 |
| 4 | 2 | 0.9% saline | 0.500 | 7.833 | 0.24 | 50 | 25 |
| 5 | 2 | 0.9% saline | 0.250 | 16.417 | 0.12 | 25 | 12.5 |
| 6 | 2 | 0.9% saline | 0.250 | 8.083 | 0.24 | 25 | 12.5 |

The samples were analysed after 0 h and the samples were stored at room temperature and analysed after the time indicated in the table below. The remaining API (i.e. the remaining melphalan flufenamide) was reported relative to the area under the curve at t=0 h. Analytical HPLC was carried out on an Agilent Series 1100 system using a Kinetex XB C18 (2.6 μm, 3.0×50 mm) column with 0.1% TFA in MiliQ H2O/MeCN as mobile phase (flow 1 mL/min). 5-97% MeCN in 3 minutes.

The results are shown in Table 9 below. Good stability at all time points was shown for all solutions comprising hydroxypropyl-γ-cyclodextrin.

TABLE 9

| Entry | Amount API (%) | Time |
|---|---|---|
| 1 | 94.3 | 18 h |
| 2 | 98.8 | 18 h |
| 3 | 97.77 | 2.5 h |
| 4 | 98.38 | 2.5 h |
| 5 | 96.10 | 2.5 h |
| 6 | 97.49 | 2.5 h |

### Example 9. Lyophilization of melphalan flufenamide in a derivative of γ-cyclodextrin, reconstitution and stability at infusion concentration

To a mixture of Ex 1 (27.1 mg, 26.0 mg free base) and hydroxypropyl-γ-cyclodextrin (1750 mg) were added t-BuOH (2000 μL) and water (3500 μL). The hydroxypropyl-γ-cyclodextrin was obtained from Sigma-Aldrich (product No 779229) and it had a degree of substitution of 0.6 (corresponding to an average number of hydroxypropyl groups per γCD molecule of about 4.7). The weight ratio (w/w) of melphalan flufenamide (free base):hydroxypropyl-γ-cyclodextrin was 1:67. The mixture was stirred for 30 min at room temperature before being lyophilized overnight. A white, fluffy lyo-cake was formed. The lyophilized material was kept refrigerated for 5 months. 0.9% saline (9000 μL) was added to the lyo-cake and the mixture was vortexed for 1 min. The material was fully dissolved to make a 3.0 mg/mL solution. HPLC analysis showed>99% purity. The reconstituted solution was diluted to 0.10 mg/mL in 0.9% saline. The stability of the solution was analyzed after 17 hours at room temperature and 91% of Ex 1 was remaining. The stability of the approved drug product PEPAXTI© at 0.10 mg/mL in 0.9% saline after 17 hours at room temperature is approximately 56%.

This example shows that when Ex 1 is lyophilised with hydroxypropyl-γ-cyclodextrin, a high reconstitution concentration can be obtained (3 mg/mL; the reconstitution concentration of the approved drug product PEPAXTI® is 0.5 mg/mL) and that good stability of was achieved after infusion concentration reconstitution with 0.9% saline.

### Example 10. Lyophilization of melphalan flufenamide in different ratios with a derivative of γ-cyclodextrin, reconstitution and stability at infusion concentration

A 10 wt % solution of hydroxypropyl-γ-cyclodextrin (1750 mg) in t-BuOH/water 1:1 (15.75 g) and a 1:1 tBuOH/water solution were prepared. The hydroxypropyl-γ-cyclodextrin was obtained from Sigma-Aldrich (product No 779229) and it had a degree of substitution of 0.6 (corresponding to an average number of hydroxypropyl groups per γCD molecule of about 4.7). Ex 1 was weighed into different vials and different amounts of the 10 wt % solution of hydroxypropyl-γ-cyclodextrin in tBuOH/water 1:1 and the 1:1 tBuOH/water solution were added according to Table 10 below to make up 4.0 mg/mL solutions of melphalan flufenamide (i.e Ex 1 as its free base). The solutions were lyophilized for 4 d.

TABLE 10

| | | | Amount | | | |
|---|---|---|---|---|---|---|
| | | | stock | | | |
| | | Ex 1, | solution of | | Amount | |
| | Ex 1- | free | 10 wt % | Amount | 50% t- | wt ratio |
| | HCl | base | HPγCD | HPγCD | BuOH:water | Ex 1, free |
| Entry | (mg) | (mg) | (mL) | (mg) | (mL) | base:HPγCD |
| 1 | 21.91 | 20.42 | 3.27 | 327 | 1.84 | 1:16 |
| 2 | 18.35 | 17.10 | 3.42 | 342 | 0.85 | 1:20 |
| 3 | 27.99 | 26.08 | 6.26 | 626 | 0.26 | 1:24 |
| 4 | 30.25 | 28.19 | 7.89 | 789 | 0.16 | 1:28 |

The lyophilized material of entries 1 to 4 of Table 10 was reconstituted with 0.9% saline to make 0.5 mg/mL, 1.0 mg/mL and 2.0 mg/mL reconstituted solutions. The lyophilized material fully dissolved in each reconstituted solution. Portions of the 0.5 mg/mL, 1.0 mg/mL and 2.0 mg/mL reconstituted solutions were then diluted to 0.10 mg/mL in 0.9% saline (infusion concentration) and these 0.10 mg/mL solutions, and also a samples of the 0.5 mg/mL reconstituted solutions, were analyzed by HPLC after 0, 4 and 20 hours at room temperature. The stability of the solutions is reported in Table 11 below as remaining Ex 1 relative to the AUC at t=0 h. The equivalents reported in Table 11 are weight equivalents to melphalan flufenamide (i.e. Ex 1 as its free base).

As can be seen from Table 11, good stability was achieved for the 0.5 mg/mL solutions, and for each 0.1 mg/mL infusion concentration reconstitution solution, at all time points, and for all w/w ratios of Ex 1:HPγCD.

TABLE 11

| | | Percentage Ex 1 remaining (%) | | | |
|---|---|---|---|---|---|
| | | 0.1 mg/mL | | 0.5 mg/mL | |
| Entry | Formulation | 4 h | 20 h | 4 h | 20 h |
| PEPAXTI ® | 50 eqv. Sucrose, | 89 | 56 | 69.9 | 16.5 |
| | 100 eqv. Glucose | | | | |
| 1 | 16 eqv. HPγCD | 96.6 | 81.3 | 97.9 | 90.1 |
| 2 | 20 eqv. HPγCD | 97.4 | 83 | 98.2 | 92.6 |
| 3 | 24 eqv. HPγCD | 97.3 | 81.9 | 98.2 | 91.4 |
| 4 | 28 eqv. HPγCD | 97.8 | 85.6 | 98.6 | 92.6 |

### Example 11. Reconstitution of melphalan flufenamide in form of EMA approved drug product, PEPAXTI®, with solutions of a derivative of γ-cyclodextrin in 5% glucose

The drug PEPAXTI®, which is approved by EMA, is a drug product consisting of melflufen hydrochloride, Ex 1 (20 mg free base) and sucrose (1000 mg) as a lyophilized product in a 50 mL vial. According to the approved protocol the drug product should be reconstituted to 0.5 mg/mL by addition of 5% glucose (40 mL). The solution is then further diluted to 0.10-0.16 mg/mL with 0.9% saline in infusion bags. In this example, the drug product PEPAXTI© (melflufen hydrochloride, Ex 1 (20 mg free base) and sucrose (1000 mg) as a lyophilized product) was reconstituted with solutions of a derivative of γ-cyclodextrin (hydroxypropyl-γ-cyclodextrin) in 5% glucose solution containing different amounts of the γ-cyclodextrin derivative.

A 5% glucose solution and a 10 wt % hydroxypropyl-γ-cyclodextrin in 5% glucose solution were prepared. The hydroxypropyl-γ-cyclodextrin was obtained from Sigma-Aldrich (product No 779229) and it had a degree of substitution of 0.6 (corresponding to an average number of hydroxypropyl groups per γCD molecule of about 4.7). One vial of PEPAXTI® drug product (melflufen hydrochloride, Ex 1 (20 mg free base) and sucrose (1000 mg) as a lyophilized product) was distributed into smaller vials and then reconstituted to 0.5 mg/mL concentration solutions using different amounts of the 5% glucose solution and the 10 wt % hydroxypropyl-γ-cyclodextrin in 5% glucose solution to give solutions having different amounts of the HPγCD excipient according to Table 12, below. The equivalents reported in Table 12 are weight equivalents to melphalan flufenamide (i.e. Ex 1 as its free base).

TABLE 12

| | | | | | 10% HPγCD | |
|---|---|---|---|---|---|---|
| | PEPAXTI ® drug | Free base | Reconstitution | Reconstitution | in 5% glucose | 5% glucose |
| Entry | product (mg) | (mg) | solution | conc. (mg/mL) | (μL) | (mL) |
| 1 | 155.4 | 3.05 | 5% glucose | 0.5 | 0 | 6.09 |
| 2 | 144.7 | 2.84 | 5% glucose + | 0.5 | 284 | 5.39 |
| | | | 10 eqv. HPγCD | | | |
| 3 | 126 | 2.47 | 5% glucose + | 0.5 | 370 | 4.57 |
| | | | 15 eqv. HPγCD | | | |
| 4 | 116.3 | 2.28 | 5% glucose + | 0.5 | 456 | 4.10 |
| | | | 20 eqv. HPγCD | | | |
| 5 | 79.1 | 1.55 | 5% glucose + | 0.5 | 388 | 2.71 |
| | | | 25 eqv. HPγCD | | | |

The 0.5 mg/mL solutions of Table 11 were then diluted with 0.9% saline to 0.16 mg/mL (320 μL+0.9% saline (680 L)) and 0.10 mg/mL (200 μL+0.9% saline (800 μL)) solutions to make the infusion concentration reconstitution solutions. These solutions were then analyzed by HPLC after 0, 4 and 20.5 h at room temperature. The stability of these solutions was reported as remaining Ex 1 relative to AUC at t=0 h. The results are shown in Table 13 below. The equivalents reported in Table 13 are weight equivalents to melphalan flufenamide (i.e.Ex 1 as its free base).

As can be seen from Table 13, improved stability was achieved at all time points for the HPγCD containing infusion concentration reconstitution solutions compared to the approved PEPAXTI® drug product reconstitution solution (5% glucose with no γCD).

TABLE 13

| | Infusion | | | | | | |
|---|---|---|---|---|---|---|---|
| | concentration | Concentration | | Concentration | | Concentration | |
| | reconstitution | 0.50 mg/mL | | 0.16 mg/mL | | 0.10 mg/mL | |
| Entry | solutions | t = 4 h | t = 20.5 h | t = 4 h | t = 20.5 h | t = 4 h | t = 20.5 h |
| 1 | 5% glucose | 69.8 | 16.4 | 88.9 | 54.1 | 89.0 | 55.9 |
| 2 | 5% glucose + | 87.5 | 51.0 | 92.3 | 67.6 | 91.9 | 65.0 |
| | 10 eqv. HPγCD | | | | | | |
| 3 | 5% glucose + | 87.6 | 52.1 | 92.2 | 67.0 | 91.6 | 61.9 |
| | 15 eqv. HPγCD | | | | | | |
| 4 | 5% glucose + | 90.6 | 61.4 | 94.6 | 73.1 | 92.9 | 68.9 |
| | 20 eqv. HPγCD | | | | | | |
| 5 | 5% glucose + | 90.6 | 63.7 | 92.7 | 73.1 | 92.7 | 69.9 |
| | 25 eqv. HPγCD | | | | | | |

### Example 12. Reconstitution of melphalan flufenamide in form of EMA approved drug product, PEPAXTI®, with solutions of a derivative of γ-cyclodextrin in 0.9% saline

As noted above, the drug PEPAXTI®, which is approved by EMA, is a drug product consisting of melflufen hydrochloride, Ex 1 (20 mg free base) and sucrose (1000 mg) as a lyophilized product in a 50 mL vial. According to the approved protocol the drug product should be reconstituted to 0.5 mg/mL by addition of 5% glucose (40 mL). The solution is then further diluted to 0.10-0.16 mg/mL with 0.9% saline in infusion bags. In this example, the drug product PEPAXTI© (melflufen hydrochloride, Ex 1 (20 mg free base) and sucrose (1000 mg) as a lyophilized product) was reconstituted with solutions of a derivative of γ-cyclodextrin (hydroxypropyl-γ-cyclodextrin) in 0.9% saline solution having different amounts of the γ-cyclodextrin derivative.

A 0.9% saline solution and a 10 wt % hydroxypropyl-γ-cyclodextrin in 0.9% saline solution were prepared. A 5 wt % hydroxypropyl-γ-cyclodextrin solution in 0.9% saline was also prepared by diluting the previous with 0.9% saline (1:1). The hydroxypropyl-γ-cyclodextrin was obtained from Sigma-Aldrich (product No 779229) and it had a degree of substitution of 0.6 (corresponding to an average number of hydroxypropyl groups per γCD molecule of about 4.7). One vial of PEPAXTI® drug product (melflufen hydrochloride, Ex 1 (20 mg free base) and sucrose (1000 mg) as a lyophilized product) was distributed into smaller vials and then reconstituted with the various saline solutions to different reconstitution concentrations with different amounts of excipient according to Table 14, below. Each vial was vortex for one minute and clear solutions were obtained. The equivalents reported in Table 14 are weight equivalents to melphalan flufenamide (i.e.Ex 1 as its free base).

TABLE 14

| | | | | | Volume | 10% | 5% | | | |
|---|---|---|---|---|---|---|---|---|---|---|
| | | Free | | Reconstitution | for | HPγCD | HPγCD | 0.9% | Weight | |
| | Amount | base | Reconstitution | concentration | reconst. | in saline | in saline | saline | eqv. | Result of |
| Entry | (mg) | (mg) | solution | (mg/mL) | (mL) | (mL) | (mL) | (mL) | HPγCD | reconstitution |
| 1 | 66.67 | 1.31 | 35 eqv. HPγCD | 0.5 | 2.613 | 0.457 | | 2.156 | 35 | soluble |
| 2 | 51.79 | 1.02 | 10% HPγCD | 0.5 | 2.030 | 2.03 | | | 200 | soluble |
| 3 | 42.02 | 0.82 | 10% HPγCD | 1 | 0.824 | 0.82 | | | 100 | soluble |
| 4 | 43.63 | 0.86 | 10% HPγCD | 1.5 | 0.570 | 0.57 | | | 67 | soluble |
| 5 | 59.64 | 1.17 | 10% HPγCD | 2 | 0.584 | 0.58 | | | 50 | soluble |
| 6 | 71.19 | 1.40 | 10% HPγCD | 2.5 | 0.558 | 0.56 | | | 40 | soluble |
| 7 | 61.33 | 1.20 | 10% HPγCD | 3 | 0.401 | 0.40 | | | 33 | soluble |
| 8 | 47.13 | 0.92 | 5% HPγCD | 1 | 0.924 | | 0.924 | | 50 | soluble |
| 9 | 41.06 | 0.80 | 5% HPγCD | 2 | 0.402 | | 0.402 | | 25 | soluble |
| 10 | 62.51 | 1.23 | 5% HPγCD | 3 | 0.408 | | 0.408 | | 17 | soluble |
| 11 | 86.87 | 1.70 | 5% HPγCD | 4 | 0.426 | | 0.426 | | 13 | soluble |

The solutions of Table 14 were then diluted with 0.9% saline to 0.10 mg/mL to make the infusion concentration reconstitution solutions. These solutions were then analyzed by HPLC after 0, 4 and 20.5 h at room temperature. The stability of these solutions was reported as remaining Ex 1 relative to AUC at t=0 h. The results are shown in Table 15 below. The equivalents reported in Table 15 are weight equivalents to melphalan flufenamide (i.e.Ex 1 as its free base).

As can be seen from Table 15, improved stability was achieved at all time points for the HPγCD containing infusion concentration reconstitution solutions compared to the approved PEPAXTI© drug product reconstituion solution (5% glucose with no γCD).

TABLE 15

| | Formulation and | Weight | | |
|---|---|---|---|---|
| | reconstitution | eqv. | | |
| Entry | concentration | HPγCD | 4 h | 20 h |
| PEPAXTI ® | 5% glucose, 0.5 mg/mL | 0 | 89 | 56 |
| current | | | | |
| protocol | | | | |
| 1 | 35 eqv. HPγCD, 0.5 mg/mL | 35 | 97.8 | 89.5 |
| 2 | 10% HPγCD, 0.5 mg/mL | 200 | 98.9 | 94.3 |
| 3 | 10% HPγCD, 1 mg/mL | 100 | 98.4 | 93.3 |
| 4 | 10% HPγCD, 1.5 mg/mL | 67 | 97.8 | 90.7 |
| 5 | 10% HPγCD, 2 mg/mL | 50 | 97.3 | 88.7 |
| 6 | 10% HPγCD, 2.5 mg/mL | 40 | 96.7 | 86.3 |
| 7 | 10% HPγCD, 3.0 mg/mL | 33 | 95.7 | not |
| | | | | recorded |
| 8 | 5% HPγCD, 1 mg/mL | 50 | 97.2 | 89.6 |
| 9 | 5% HPγCD, 2 mg/mL | 25 | 95.2 | 84.3 |
| 10 | 5% HPγCD, 3 mg/mL | 17 | 94.5 | 80.6 |
| 11 | 5% HPγCD, 4 mg/mL | 12 | 93.5 | 77.5 |

### Example 13. Lyophilized material with and without sucrose and a derivate of γ-cyclodextrin

A solution of Ex 1 (121.7 mg, 113 mg free base) in t-BuOH/water 1:1 (22.6 mL) was prepared to make a stock solution of 5 mg/mL Ex 1. Aliquots of (400 μL, 2.00 mg Ex 1 as free base) were distributed to tubes. A 10% solution of sucrose in water and a solution of 10% hydroxypropyl-γ-cyclodextrin in water were prepared. The hydroxypropyl-γ-cyclodextrin was obtained from Sigma-Aldrich (product No 779229) and it had a degree of substitution of 0.6 (corresponding to an average number of hydroxypropyl groups per γCD molecule of about 4.7). To the tubes were added excipients according to the table below. The samples were then lyophilized for 4 d.

TABLE 16

| | | | | 10% | 10% | Extra t- |
|---|---|---|---|---|---|---|
| | | Free | | HPγCD | sucrose | BuOH/water |
| | Ex 1 | base | | in water | in water | (1:1) added |
| Entry | (mg) | (mg) | mmol | (μL) | (μL) | (mL) |
| 1 | 2.15 | 2.00 | 0.0040 | | 963 | 1 |
| 2 | 2.15 | 2.00 | 0.0040 | 320 | 963 | 1 |
| 3 | 2.15 | 2.00 | 0.0040 | 320 | | |

For entry 1, the lyophilized sample was reconstituted with 5% glucose (4 mL) and the vial vortexed for 1 min. Entries 2 and 3 were reconstituted with 0.9% saline (1 mL) and the vials vortexed for 1 min. The samples were diluted to 0.10 mg/mL in 0.9% saline. These samples were then analyzed by HPLC after 0, 4 and 23 h at room temperature. The stability of these solutions was reported in Table 17, below, as remaining Ex 1 relative to AUC at t=0 h. The equivalents reported in Table 17 are weight equivalents to melphalan flufenamide (i.e.Ex 1 as its free base). The best stability at all time points was observed for the solutions including the hydroxypropyl-γ-cyclodextrin.

TABLE 17

| | | | Ex 1 | Ex 1 |
|---|---|---|---|---|
| | | Reconstitution | remaining | remaining |
| | | concentation | after | after 23 h |
| Entry | Formulation | (mg/mL) | 4 h (%) | (%) |
| 1 | Melflufen/sucrose/ | 0.5 | 89.6 | 54.1 |
| | HPγCD 1:50:0 | | | |
| | (PEPAXTI ®) | | | |
| 2 | Melflufen/sucrose/ | 4 | 93.4 | 66.6 |
| | HPγCD 1:50:16 | | | |
| 3 | Melflufen/sucrose/ | 4 | 91.6 | 63.7 |
| | HPγCD 1:0:16. | | | |

1. A pharmaceutical preparation comprising melphalan flufenamide, or a salt thereof, and γ-cyclodextrin or a pharmaceutically acceptable derivative of γ-cyclodextrin.

2. The pharmaceutical preparation of claim 1, wherein the weight ratio (w/w) between the γ-cyclodextrin or a pharmaceutically acceptable derivative of γ-cyclodextrin and melphalan flufenamide is 1:1 to 100:1, 4:1 to 80:1, 5:1 to 25:1, or 10:1 to 25:1.

3. The pharmaceutical preparation of claim 1 or 2, wherein said pharmaceutical preparation is a lyophilized powder.

4. The pharmaceutical preparation of claim 3, wherein the pharmaceutical preparation comprises 1 mg to 30 mg melphalan flufenamide (excluding the mass of any counterion).

5. The pharmaceutical preparation of claim 4, comprising sucrose.

6. The pharmaceutical preparation of claim 5, wherein the weight ratio (w/w) of sucrose to melphalan flufenamide is 25:1 to 75:1, for example 50:1.

7. The pharmaceutical preparation of any one of claims 1 to 6, which is substantially free from organic solvent.

8. The pharmaceutical preparation of any one of claims 1 or 2, wherein said pharmaceutical preparation is a liquid formulation comprising propylene glycol.

9. The pharmaceutical preparation of claim 8, wherein said melphalan flufenamide, or salt thereof, is at a concentration of about 10 mg/mL to about 50 mg/mL (excluding the mass of any counterion).

10. The pharmaceutical preparation of claim 9, wherein said melphalan flufenamide, or salt thereof, is at a concentration of about 15 mg/mL to about 25 mg/mL (excluding the mass of any counterion).

11. The pharmaceutical preparation of any one of claims 1 to 10, comprising one or more additional therapeutic agent(s) selected from the group consisting of a protease inhibitor (PI), an immunomodulatory drug (IMiD), an alkylator and a monoclonal antibody (for example an anti-CD38 antibody or an anti-SLAMF7 antibody).

12. The pharmaceutical preparation of any one of claims 3 to 7, wherein said pharmaceutical preparation consists essentially of melphalan flufenamide, or a salt thereof, γ-cyclodextrin or a pharmaceutically acceptable derivative of γ-cyclodextrin, and sucrose.

13. The pharmaceutical preparation of any one of claims 8 to 10, wherein said pharmaceutical preparation consists essentially of melphalan flufenamide, or a salt thereof; γ-cyclodextrin or a pharmaceutically acceptable derivative of γ-cyclodextrin; and propylene glycol and/or PEG.

14. The pharmaceutical preparation of any one of claims 1 to 13, wherein the melphalan flufenamide is in the form of its hydrochloride salt.

15. The pharmaceutical preparation of any one of claims 1 to 14, wherein the melphalan flufenamide is deuterated melphalan flufenamide, preferably the deuterated melphalan flufenamide has the structure

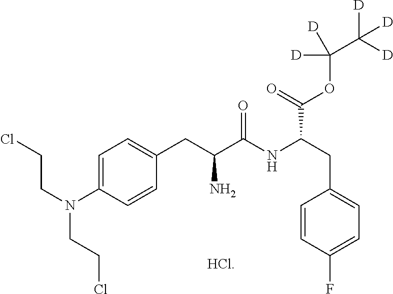 [SMILES: [2H]C([2H])([2H])C([2H])([2H])OC(=O)[C@H](Cc1ccc(F)cc1)NC(=O)[C@@H](N)Cc1ccc(N(CCCl)CCCl)cc1 ; "HCl."]

16. The pharmaceutical preparation of any one of claims 1 to 15, wherein the pharmaceutically acceptable derivative of γ-cyclodextrin is selected from the group consisting of hydroxypropyl-γ-cyclodextrin, succinyl-γ-cyclodextrin, sulfobutyl ether-γ-cyclodextrin, γ-cyclodextrin phosphate sodium salt, γ-cyclodextrin sulphate sodium salt, 2-carboxyethyl-γ-cyclodextrin, acetylated-γ-cyclodextrin, and sugammadex; and preferably wherein the pharmaceutically acceptable derivative of γ-cyclodextrin is hydroxypropyl-γ-cyclodextrin.

17. The pharmaceutical preparation of any one of claims 1 to 16, wherein the weight ratio (w/w) between the γ-cyclodextrin or a pharmaceutically acceptable derivative of γ-cyclodextrin and melphalan flufenamide is 15:1 to 25:1, for example 20:1 to 25:1.

18. A composition comprising a pharmaceutical preparation of any of claims 1 to 17 and a physiologically acceptable aqueous solvent or diluent.

19. A composition comprising melphalan flufenamide, or a salt thereof, γ-cyclodextrin or a pharmaceutically acceptable derivative of γ-cyclodextrin, and a physiologically acceptable aqueous solvent.

20. The composition of claim 18 or claim 19, wherein the physiologically acceptable aqueous solvent is a glucose solution, a saline solution, or a mixture thereof.

21. A kit comprising a pharmaceutical preparation of any of claims 1 to 17 and a physiologically acceptable aqueous solvent or diluent.

22. A kit comprising melphalan flufenamide, or a salt(s) thereof, γCD or a pharmaceutically acceptable derivative of γ-cyclodextrin, and optionally one or more physiologically acceptable aqueous solvent or diluent.

23. The kit of claim 21 wherein the melphalan flufenamide, or a salt(s) thereof, is provided as a lyophilised preparation comprising (or consisting essentially of) melphalan flufenamide and sucrose, or provided as a liquid pharmaceutical formulation comprising (or consisting essentially of) melphalan flufenamide and propylene glycol and/or polyethylene glycol.

24. The pharmaceutical preparation of claims 1 to 17, or the composition of claims 18, 19 or 20, or the kit of claims 21, 22 or 23, for use as a medicament.

25. The pharmaceutical preparation of any one of claims 1 to 17, or the composition of claims 18, 19 or 20, or the kit of claims 21, 22 or 23, for use in treatment or prophylaxis of a disease or condition that may be treated with conventional chemotherapy agents, for example, with an alkylator (e.g. melphalan, cyclophosphamide and bendamustine).

26. The pharmaceutical preparation of any one of claims 1 to 17, or the composition of claims 18, 19 or 20, or the kit of claims 21, 22 or 23, for use in the treatment or prophylaxis of cancer or for use in the treatment or prophylaxis of amyloidosis.

27. The pharmaceutical preparation of any one of claims 1 to 17, or the composition of claims 18, 19 or 20, or the kit of claims 21, 22 or 23, for use in the treatment or prophylaxis of cancer wherein the cancer is selected from the group consisting of multiple myeloma, breast cancer, lung cancer, ovarian cancer, glioblastoma, leukaemias and lymphomas.

28. A method for treating a patient which comprises administering a pharmaceutically effective amount of the pharmaceutical preparation of any one of claims 1 to 17 or the composition of claims 18, 19 or 20 to a subject in need thereof.

29. A method for the treatment or prophylaxis of cancer, comprising administering an effective amount of the pharmaceutical preparation of any one of claims 1 to 17, or the composition of claims 18, 19 or 20, to a subject in need thereof.
